SEQUENCE LISTING (69 sequences)

SEQ ID NO: 1 - protein, 107 aa, Artificial sequence
  synthetic sequence
DIVLTQSPSSFSVSLGDRVTISCKASGYILNRLAWYQQKPGNAPRLLISGATSLETGFPSRFSGTGSGKDYTLSISSLQTEDVGTYYCQQYWSTPWTFGGGTKLEIR

SEQ ID NO: 2 - protein, 11 aa, Artificial sequence
  synthetic sequence
KASGYILNRLA

SEQ ID NO: 3 - protein, 7 aa, Artificial sequence
  synthetic sequence
GATSLET

SEQ ID NO: 4 - protein, 9 aa, Artificial sequence
  synthetic sequence
QQYWSTPWT

SEQ ID NO: 5 - protein, 119 aa, Artificial sequence
  synthetic sequence
QVQLQESGPGLVKPSQSLSLTCTVTGFSITSDYAWNWIRQFPGKKLEWMGYINFDGGTTYNPSLRGRISITRDTSKNQFFLQLRSVTPEDTATYYCATFYGAKGTLDYWGQGTSVTVSS

SEQ ID NO: 6 - protein, 119 aa, Artificial sequence
  synthetic sequence
EVQLQESGPGLVKPSETLSLTCTVSGFSITSDYAWNWIRQPPGKGLEWMGYINFDGGTTYNPSLRGRITISRDTSKNQFSLKLSSVTAADTAVYYCATFYGAKGTLDYWGQGTLVTVSS

SEQ ID NO: 7 - protein, 119 aa, Artificial sequence
  synthetic sequence
EVQLQESGPGLVKPSETLSLTCTVSGFSITSDYAWNWIRQPPGKGLEWIGYINFDGGTTYNPSLRGRVTISRDTSKNQFSLKLSSVTAADTAVYYCATFYGAKGTLDYWGQGTLVTVSS

SEQ ID NO: 8 - protein, 117 aa, Artificial sequence
  synthetic sequence
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 9 - protein, 5 aa, Artificial sequence
  synthetic sequence
NYNMH

SEQ ID NO: 10 - protein, 17 aa, Artificial sequence
  synthetic sequence
TIYPGNDDTSYNQKFKD

SEQ ID NO: 11 - protein, 8 aa, Artificial sequence
  synthetic sequence
GGYRAMDY

SEQ ID NO: 12 - protein, 119 aa, Artificial sequence [sequence omitted]

SEQ ID NO: 13 - protein, 5 aa, Artificial sequence
  synthetic sequence
DYTMD

SEQ ID NO: 14 - protein, 17 aa, Artificial sequence
  synthetic sequence
DVNPNSGGSIYNQRFKG

SEQ ID NO: 15 - protein, 10 aa, Artificial sequence
  synthetic sequence
NLGPSFYFDY

SEQ ID NO: 16 - protein, 448 aa, Artificial sequence
  synthetic sequence
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 451 aa, Artificial sequence
  synthetic sequence
QVQLQESGPGLVKPSQTLSLTCTVSGGSISSGDYYWSWIRQPPGKGLEWIGYIYYSGSTDYNPSLKSRVTMSVDTSKNQFSLKVNSVTAADTAVYYCARVSIFGVGTFDYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 449 aa, Artificial sequence
  synthetic sequence
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 7 aa, Artificial sequence
  synthetic sequence
SGDYYWS

SEQ ID NO: 20 - protein, 16 aa, Artificial sequence
  synthetic sequence
YIYYSGSTDYNPSLKS

SEQ ID NO: 21 - protein, 11 aa, Artificial sequence
  synthetic sequence
VSIFGVGTFDY

SEQ ID NO: 22 - protein, 121 aa, Artificial sequence [sequence omitted]

SEQ ID NO: 23 - protein, 5 aa, Artificial sequence
  synthetic sequence
NYGVH

SEQ ID NO: 24 - protein, 16 aa, Artificial sequence
  synthetic sequence
VIWSGGNTDYNTPFTS

SEQ ID NO: 25 - protein, 11 aa, Artificial sequence
  synthetic sequence
ALTYYDYEFAY

SEQ ID NO: 26 - protein, 119 aa, Artificial sequence
  synthetic sequence
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 27 - protein, 118 aa, Artificial sequence [sequence omitted]

SEQ ID NO: 28 - protein, 5 aa, Artificial sequence
  synthetic sequence
DSWIH

SEQ ID NO: 29 - protein, 17 aa, Artificial sequence
  synthetic sequence
WISPYGGSTYYADSVKG

SEQ ID NO: 30 - protein, 9 aa, Artificial sequence
  synthetic sequence
RHWPGGFDY

SEQ ID NO: 31 - protein, 448 aa, Artificial sequence
  synthetic sequence
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 655 aa, Homo sapiens
MSSSNVEVFIPVSQGNTNGFPATASNDLKAFTEGAVLSFHNICYRVKLKSGFLPCRKPVEKEILSNINGIMKPGLNAILGPTGGGKSSLLDVLAARKDPSGLSGDVLINGAPRPANFKCNSGYVVQDDVVMGTLTVRENLQFSAALRLATTMTNHEKNERINRVIQELGLDKVADSKVGTQFIRGVSGGERKRTSIGMELITDPSILFLDEPTTGLDSSTANAVLLLLKRMSKQGRTIIFSIHQPRYSIFKLFDSLTLLASGRLMFHGPAQEALGYFESAGYHCEAYNNPADFFLDIINGDSTAVALNREEDFKATEIIEPSKQDKPLIEKLAEIYVNSSFYKETKAELHQLSGGEKKKKITVFKEISYTTSFCHQLRWVSKRSFKNLLGNPQASIAQIIVTVVLGLVIGAIYFGLKNDSTGIQNRAGVLFFLTTNQCFSSVSAVELFVVEKKLFIHEYISGYYRVSSYFLGKLLSDLLPMRMLPSIIFTCIVYFMLGLKPKADAFFVMMFTLMMVAYSASSMALAIAAGQSVVSVATLLMTICFVFMMIFSGLLVNLTTIASWLSWLQYFSIPRYGFTALQHNEFLGQNFCPGLNATGNNPCNYATCTGEEYLVKQGIDLSPWGLWKNHVALACMIVIFLTIAYLKLLFLKKYS

SEQ ID NO: 33 - protein, 12 aa, Homo sapiens
KNDSTGIQNRAG

SEQ ID NO: 34 - protein, 8 aa, Homo sapiens
LKPKADAF

SEQ ID NO: 35 - protein, 74 aa, Homo sapiens
NLTTIASWLSWLQYFSIPRYGFTALQHNEFLGQNFCPGLNATGNNPCNYATCTGEEYLVKQGIDLSPWGLWKNH

SEQ ID NO: 36 - protein, 657 aa, Mus musculus
MSSSNDHVLVPMSQRNNNGLPRTNSRAVRTLAEGDVLSFHHITYRVKVKSGFLVRKTVEKEILSDINGIMKPGLNAILGPTGGGKSSLLDVLAARKDPKGLSGDVLINGAPQPAHFKCCSGYVVQDDVVMGTLTVRENLQFSAALRLPTTMKNHEKNERINTIIKELGLEKVADSKVGTQFIRGISGGERKRTSIGMELITDPSILFLDEPTTGLDSSTANAVLLLLKRMSKQGRTIIFSIHQPRYSIFKLFDSLTLLASGKLVFHGPAQKALEYFASAGYHCEPYNNPADFFLDVINGDSSAVMLNREEQDNEANKTEEPSKGEKPVIENLSEFYINSAIYGETKAELDQLPGAQEKKGTSAFKEPVYVTSFCHQLRWIARRSFKNLLGNPQASVAQLIVTVILGLIIGAIYFDLKYDAAGMQNRAGVLFFLTTNQCFSSVSAVELFVVEKKLFIHEYISGYYRVSSYFFGKVMSDLLPMRFLPSVIFTCVLYFMLGLKKTVDAFFIMMFTLIMVAYTASSMALAIATGQSVVSVATLLMTIAFVFMMLFSGLLVNLRTIGPWLSWLQYFSIPRYGFTALQYNEFLGQEFCPGFNVTDNSTCVNSYAICTGNEYLINQGIELSPWGLWKNHVALACMIIIFLTIAYLKLLFLKKYS

SEQ ID NO: 37 - protein, 14 aa, Mus musculus
DLKYDAAGMQNRAG

SEQ ID NO: 38 - protein, 8 aa, Mus musculus
LKKTVDAF

SEQ ID NO: 39 - protein, 76 aa, Mus musculus
NLRTIGPWLSWLQYFSIPRYGFTALQYNEFLGQEFCPGFNVTDNSTCVNSYAICTGNEYLINQGIELSPWGLWKNH

SEQ ID NO: 40 - protein, 655 aa, Macaca fascicularis
MSSSNVEVFIPMSQENTNGFPTTTSNDRKAFTEGAVLSFHNICYRVKVKSGFLPGRKPVEKEILSNINGIMKPGLNAILGPTGGGKSSLLDVLAARKDPSGLSGDVLINGALRPTNFKCNSGYVVQDDVVMGTLTVRENLQFSAALRLPTTMTNHEKNERINRVIQELGLDKVADSKVGTQFIRGVSGGERKRTSIGMELITDPSILFLDEPTTGLDSSTANAVLLLLKRMSKQGRTIIFSIHQPRYSIFKLFDSLTLLASGRLMFHGPAQEALGYFESAGYHCEAYNNPADFFLDIINGDSTAVALNREEDFKATEIIEPSKRDKPLVEKLAEIYVDSSFYKETKAELHQLSGGEKKKKITVFKEISYTTSFCHQLRWVSKRSFKNLLGNPQASIAQIIVTVILGLVIGAIYFGLNNDSTGIQNRAGVLFFLTTNQCFSSVSAVELFVVEKKLFIHEYISGYYRVSSYFFGKLLSDLLPMRMLPSIIFTCIVYFMLGLKPTADAFFIMMFTLMMVAYSASSMALAIAAGQSVVSVATLLMTICFVFMMIFSGLLVNLTTIASWLSWLQYFSIPRYGFTALQHNEFLGQNFCPGLNATVNNTCNYATCTGEEYLTKQGIDLSPWGLWKNHVALACMIVIFLTIAYLKLLFLKKYS

SEQ ID NO: 41 - protein, 12 aa, Macaca fascicularis
NNDSTGIQNRAG

SEQ ID NO: 42 - protein, 8 aa, Macaca fascicularis
LKPTADAF

SEQ ID NO: 43 - protein, 74 aa, Macaca fascicularis
NLTTIASWLSWLQYFSIPRYGFTALQHNEFLGQNFCPGLNATVNNTCNYATCTGEEYLTKQGIDLSPWGLWKNH

SEQ ID NO: 44 - protein, 655 aa, Pan troglodytes
MSSSNVEVFIPMSQGNTNGFPATTSNDLKAFTEGAVLSFHNICYRVKLKSGFLPCRKPVEKEILSNINGIMKPGLNAILGPTGGGKSSLLDVLAARKDPSGLSGDVLINGAPRPANFKCNSGYVVQDDVVMGTLTVRENLQFSAALRLPTTMTNHEKNERINRVIQELGLDKVADSKVGTQFIRGVSGGERKRTSIGMELITDPSILFLDEPTTGLDSSTANAVLLLLKRMSKQGRTIIFSIHQPRYSIFKLFDSLTLLASGRLMFHGPAQEALGYFESAGYHCEAYNNPADFFLDIINGDSTAVALNREEDFKATEIIEPSKQDKPLIEKLAEIYVNSSFYKETKAELHQLSGGEKKKKITVFKEISYTTSFCHQLRWVSKRSFKNLLGNPQASIAQIIVTVILGLVIGAIYFGLKNDSTGIQNRAGVLFFLTTNQCFSSVSAVELFVVEKKLFIHEYISGYYRVSSYFLGKLLSDLLPMRMLPSIIFTCIVYFMLGLKPKADAFFVMMFTLMMVAYSASSMALAIAAGQSVVSVATLLMTICFVFMMIFSGLLVNLTTIASWLSWLQYFSIPRYGFTALQHNEFLGQNFCPGLNATGNNPCNYATCTGEEYLVKQGIDLSPWGLWKNHVALACMIVIFLTIAYLKLLFLKKYS

SEQ ID NO: 45 - protein, 655 aa, Homo sapiens
MSSSNVEVFIPVSQGNTNGFPATASNDLKAFTEGAVLSFHNICYRVKLKSGFLPCRKPVEKEILSNINGIMKPGLNAILGPTGGGKSSLLDVLAARKDPSGLSGDVLINGAPRPANFKCNSGYVVQDDVVMGTLTVRENLQFSAALRLATTMTNHEKNERINRVIQELGLDKVADSKVGTQFIRGVSGGERKRTSIGMELITDPSILFLDQPTTGLDSSTANAVLLLLKRMSKQGRTIIFSIHQPRYSIFKLFDSLTLLASGRLMFHGPAQEALGYFESAGYHCEAYNNPADFFLDIINGDSTAVALNREEDFKATEIIEPSKQDKPLIEKLAEIYVNSSFYKETKAELHQLSGGEKKKKITVFKEISYTTSFCHQLRWVSKRSFKNLLGNPQASIAQIIVTVVLGLVIGAIYFGLKNDSTGIQNRAGVLFFLTTNQCFSSVSAVELFVVEKKLFIHEYISGYYRVSSYFLGKLLSDLLPMRMLPSIIFTCIVYFMLGLKPKADAFFVMMFTLMMVAYSASSMALAIAAGQSVVSVATLLMTICFVFMMIFSGLLVNLTTIASWLSWLQYFSIPRYGFTALQHNEFLGQNFCPGLNATGNNPCNYATCTGEEYLVKQGIDLSPWGLWKNHVALACMIVIFLTIAYLKLLFLKKYS

SEQ ID NO: 46 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 47 - protein, 303 aa, Mus musculus
MWPLAAALLLGSCCCGSAQLLFSNVNSIEFTSCNETVVIPCIVRNVEAQSTEEMFVKWKLNKSYIFIYDGNKNSTTTDQNFTSAKISVSDLINGIASLKMDKRDAMVGNYTCEVTELSREGKTVIELKNRTVSWFSPNEKILIVIFPILAILLFWGKFGILTLKYKSSHTNKRIILLLVAGLVLTVIVVVGAILLIPGEKPVKNASGLGLIVISTGILILLQYNVFMTAFGMTSFTIAILITQVLGYVLALVGLCLCIMACEPVHGPLLISGLGIIALAELLGLVYMKFVASNQRTIQPPRNR

SEQ ID NO: 48 - protein, 323 aa, Pongo abelii
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLIITVIVIVGAILFVPGEYSLKNATGLGLIVTSTGILILLHYYVFSTAIGLNSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 49 - protein, 323 aa, Macaca mulatta
MWPLVAALLLGSACCGSAQLLFNKTKSVEFTFCNDTVVIPCFVTNMEAQNTTEVYVKWKFKGRDIYTFDGALNKSTAPANFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 50 - protein, 446 aa, Artificial sequence
  synthetic sequence
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 51 - protein, 5 aa, Artificial sequence
  synthetic sequence
  REPEAT (1)..(5): The sequence may repeat n times, where n is an integer of at least one
GSGGS

SEQ ID NO: 52 - protein, 4 aa, Artificial sequence
  synthetic sequence
  REPEAT (1)..(4): The sequence may repeat n times, where n is an integer of at least one
GGGS

SEQ ID NO: 53 - protein, 4 aa, Artificial sequence
  synthetic sequence
GGSG

SEQ ID NO: 54 - protein, 5 aa, Artificial sequence
  synthetic sequence
GGSGG

SEQ ID NO: 55 - protein, 5 aa, Artificial sequence
  synthetic sequence
GSGSG

SEQ ID NO: 56 - protein, 5 aa, Artificial sequence
  synthetic sequence
GSGGG

SEQ ID NO: 57 - protein, 5 aa, Artificial sequence
  synthetic sequence
GGGSG

SEQ ID NO: 58 - protein, 5 aa, Artificial sequence
  synthetic sequence
GSSSG

SEQ ID NO: 59 - protein, 107 aa, Artificial sequence
  synthetic sequence
DIQLTQSPSSLSASVGDRVTITCKASGYILNRLAWYQQKPGKAPKLLISGATSLETGFPSRFSGSGSGKDYTLTISSLQPEDFATYYCQQYWSTPWTFGGGTKLEIK

SEQ ID NO: 60 - protein, 111 aa, Artificial sequence
  synthetic sequence
DIVMTQSPLSLPVTPGEPASISCRSSQSIVYSNGNTYLGWYLQKPGQSPQLLIYKVSNRFSGVPDRFSGSGSGTDFTLKISRVEADVGVYYCFQGSHVPYTFGQGTKLEIK

SEQ ID NO: 61 - protein, 214 aa, Artificial sequence
  synthetic sequence
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 214 aa, Artificial sequence
  synthetic sequence
EIVMTQSPATLSLSPGERATLSCRASQSVSSYLAWYQQKPGQAPRLLIYDASNRATGIPARFSGSGSGTDFTLTISSLEPEDFAVYYCHQYGSTPLTFGGGTKAEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 6 aa, Artificial sequence
  synthetic sequence
SDYAWN

SEQ ID NO: 64 - protein, 16 aa, Artificial sequence
  synthetic sequence
YINFDGGTTYNPSLRG

SEQ ID NO: 65 - protein, 10 aa, Artificial sequence
  synthetic sequence
FYGAKGTLDY

SEQ ID NO: 66 - protein, 117 aa, Artificial sequence
  synthetic sequence
EVQLVESGGGLVQPGGSLRLSCAASGYTFTNYNMHWVRQAPGKGLEWMGTIYPGNDDTSYNQKFKDRVTISRDNSKNTLYLQMNSLRAEDTAVYYCARGGYRAMDYWGQGTLVTVSS

SEQ ID NO: 67 - protein, 117 aa, Artificial sequence
  synthetic sequence
EVQLVQSGAEVKKPGESLKISCKGSGYTFTNYNMHWVRQMPGKGLEWMGTIYPGNDDTSYNQKFKDQVTISADKSISTAYLQWSSLKASDTAMYYCARGGYRAMDYWGQGTTVTVSS

SEQ ID NO: 68 - protein, 117 aa, Artificial sequence
  synthetic sequence
QVQLVQSGSELKKPGASVKVSCKASGYTFTNYNMHWVRQAPGQGLEWMGTIYPGNDDTSYNQKFKDRFVFSLDTSVSTAYLQISSLKAEDTAVYYCARGGYRAMDYWGQGTTVTVSS

SEQ ID NO: 69 - protein, 10 aa, Artificial sequence
  synthetic sequence
  SITE (3)..(10): The amino acids at positions 3 to 10 may independently either be present or absent
GGGGGGGGGG

(19) United States
(12) Patent Application Publication
(10) Pub. No.: US 2023/0192902 A1
(43) Pub. Date: Jun. 22, 2023

(54) ABCG2 EFFLUX PUMP-CANCER ANTIGEN MULTI-SPECIFIC ANTIBODIES AND COMPOSITIONS, REAGENTS, KITS AND METHODS RELATED THERETO

(71) Applicant: Kenjockety Biotechnology, Inc., Tiburon, CA (US)

(72) Inventors: William Robert Arathoon, Tiburon, CA (US); Raffaella Briante, Burlingame, CA (US); Suchismita Mohanty, San Bruno, CA (US); Paul David Ponath, San Francisco, CA (US); Qianting Zhai, South San Francisco, CA (US)

(21) Appl. No.: 17/998,841

(22) PCT Filed: May 28, 2021
(86) PCT No.: PCT/US2021/034913

§ 371 Date: Nov. 15, 2022

Related U.S. Application Data
(60) Provisional application No. 63/034,822, filed on Jun. 4, 2020.

(51) Int. Cl. C07K 16/46; A61P 35/00

(52) U.S. Cl. CPC C07K 16/468; A61P 35/00; C07K 2317/24; C07K 2317/31; C07K 2317/52; C07K 2317/565

(57) ABSTRACT

Provided are multi-specific antibodies that target both the cellular efflux pump ABCG2 and a cancer-associated antigen as well as pharmaceutical compositions, nucleic acids, recombinant expression vectors, cells, and kits that include or encode such multi-specific antibodies. Methods of treating a subject for a cancer that include administering to the subject a multi-specific antibody that targets both a cellular efflux pump and a cancer-associated antigen are also provided. Provided as well are methods of generating the described multi-specific antibodies and reagents related thereto, including genetically modified cell lines useful in the subject methods and methods of making such genetically modified cell lines.

### CROSS-REFERENCE TO RELATED APPLICATIONS

This application claims the benefit of priority to U.S. Provisional Pat. Application No. 63/034,822 filed on Jun. 4, 2020, which application is incorporated herein by reference in its entirety.

### INCORPORATION BY REFERENCE OF SEQUENCE LISTING PROVIDED AS TEXT FILE

A Sequence Listing is provided herewith as a text file, “KNJY-005WO SEQ LIST_ST25.txt,” created on May 27, 2021 and having a size of 87 KB. The contents of the text file are incorporated by reference herein in their entirety.

### INTRODUCTION

Drug resistance, a well-known phenomenon that results when diseases become tolerant to pharmaceutical treatments, is a major and increasing challenge in various fields of medicine, including oncology. Although many types of cancers are initially susceptible to chemotherapy, over time they can develop resistance through these and other mechanisms, including DNA mutations and metabolic changes that promote drug inhibition, degradation and enhanced efflux.

Efflux pumps (EP) are proteins expressed by living cells and have evolved to naturally expel various compounds from the cells. Members of the ATP-binding cassette (ABC) transporter family proteins are examples of EPs that enable drug efflux. Though a transporter’s structure varies from protein to protein (e.g., there are 49 known members of the ABC family in humans), they are all classified by the presence of two distinct domains-a highly conserved nucleotide binding domain and a more variable transmembrane domain. Multidrug resistance protein 1 (ABCG2), encoded by the ATP Binding Cassette Subfamily B Member 1 (ABCB1) gene, was the first of these to be identified and has been studied extensively. ABCG2 expression is increased in response to treatment with certain chemotherapeutics.

EPs enable tumors to develop resistance to chemotherapeutic agents. Such resistance is frequently associated with enhanced efflux of the chemotherapeutic agent from the drug resistant cells. This chemotherapy resistance is termed multi drug resistance (MDR) when it applies to more than one chemotherapeutic agent.

Among cancer patients, where metastatic cancer cell populations have largely been killed and cleared from the patients by use of chemotherapy, it is not uncommon for a drug-resistant cancerous population of cells to emerge and spread without response to renewed treatment with the earlier therapy. In most cases, a different drug with a different mechanism of action is applied until, once again, another emergent population of drug-resistant cells and/or tumor develops.

As such there is a need to develop reagents that may be used for inhibiting EPs.

### SUMMARY

Provided are anti-ABCG2 antibodies that may be used as multi-specific antibodies that target both ABCG2 and a tumor-associated antigen (TAA) as well as pharmaceutical compositions, nucleic acids, recombinant expression vectors, cells, and kits that include or encode such multi-specific antibodies. The multi-specific antibodies include a common variable light (VL) chain that includes an antigen-binding site for ABCG2, a first variable heavy (VH) chain that includes an antigen-binding site for ABCG2, and a second VH chain that includes an antigen-binding site for the TAA or a first VH chain and a first VL chain that include an antigen-binding site for ABCG2 and a second VH chain and a second VL chain that include an antigen-binding site for the TAA. Methods of treating a subject for a cancer that include administering to the subject a multi-specific antibody that targets both ABCG2 and a TAA are also provided. The treating may involve administering the multi-specific antibody alone or administering the multi-specific antibody and a chemotherapeutic agent. Provided as well are methods of generating the described multi-specific antibodies and reagents related thereto, including genetically modified cell lines useful in the subject methods and methods of making such genetically modified cell lines.

The bispecific antibodies provided herein bind to cancer cells expressing both ABCG2 and the TAA while showing reduced binding to non-cancer cells expressing ABCG2 and/or the TAA. In other words, the bispecific antibodies provided herein bind with low affinity to (1) cells expressing TAA where ABCG2 expression is low or absent and (2) cells expressing ABCG2 where TAA expression is low or absent, and with high affinity to cancer cells that express at least one or both ABCG2 and TAA at relatively high levels, i.e., levels higher than normal cells.

### BRIEF DESCRIPTION OF THE FIGURES

FIG. 1 provides chemosensitivity analysis showing 293T cells overexpressing ABCG2 have increased susceptibility to topotecan in the presence of the anti-ABCG2 antibody 5D3 as well as a bispecific antibody that binds to ABCG2 and CD47. The bispecific antibody 5D3DDKT14KK5D3 includes heavy chain (HC) and light chain (LC) from the anti-ABCG2 antibody 5D3 and a HC from the anti-CD47 antibody, 5F9. The bispecific antibody 5D3hVHv1DDKT14KK5D3hVLv1 includes HC and LC from the anti-ABCG2 antibody 5D3 and a HC from the anti-CD47 antibody, 5F9. The bispecific antibody 5D3hVHv2DDKT14KK5D3hVLv1 includes HC and LC from the anti-ABCG2 antibody 5D3 and a HC from the anti-CD47 antibody, 5F9. hVHv1 and hVHv2 refer to humanized variable heavy chain version 1 and version 2, respectively, of the 5D3 HC. hVLv1 refers to humanized variable light chain version 1 of the 5D3 LC. KT14 refers to CD47. DD and KK refer to charged pair substitutions that enhance pairing between 5D3 and 5F9 HCs. IC50 values are in nM.

FIG. 2 shows the binding of 5D3 and humanized 5D3 antibodies to 293T-G2OX cells along with the corresponding EC50 binding affinities. EC50 values are in nM.

FIG. 3 shows the binding of humanized ABCG2/KT9 bispecific antibodies to 3T3 cells stably transfected to express ABCG2 (3T3-G2), 293T cells stably transfected to express human ABCG2 (293T_ABCG2_OX), and 293T cells stably transfected to express human KT9 (293T-KT9OX). KT9 is atezolizumab, an anti-PD-L1 antibody, 5D3 is an anti-ABCG2 antibody. The humanized bispecific antibodies 5D3hVH-v1DD KT9KK 5D3hVL-v1 and 5D3hVH-v2DD KT9KK 5D3hVL-v1 include HC and LC from the anti-ABCG2 antibody 5D3 and a HC from the anti-PD-L1 antibody, KT9.

FIG. 4 shows the binding of humanized 5D3/KT9/5D3 bispecific antibodies to 293T cells stably transfected with ABCG2 (293T-G2OX), KT9 (293T-KT9OX) and both ABCG2 and KT9 (293T-G2KT9OX), along with the corresponding EC50 binding affinity values.

FIG. 5 shows the results of a xenograft study where the cytotoxic activity of an ABCG2/PD-L1 bispecific antibody (5D3/KT9), alone or in combination with topotecan, was tested in the HT1376 (ATCC CRL-1472) human urinary bladder epithelial carcinoma cell line.

### DEFINITIONS

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, including antibodies comprising only heavy chains (e.g. VHH camelid antibodies), bispecific antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the term are Fab′, Fv, F(ab′)₂, and or other antibody fragments that retain specific binding to antigen, and monoclonal antibodies. An antibody may be monovalent or bivalent. The antibodies used herein may be used to assay expression of a target antigens(s) on a cell surface, e.g., in a cell sample or a tissue sample from a patient.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); single-chain antibody molecules, including antibodies comprising only heavy chains (e.g. VHH camelid antibodies); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen and can form the antigen binding site, although at a lower affinity than the entire binding site comprising the three CDRs of each variable domain.

The “Fab” fragment also contains the constant domain of the light chain and the first constant domain (CH₁) of the heavy chain. Fab fragments differ from Fab′ fragments by the addition of a few residues at the carboxyl terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv”, “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. A ABCG2-specific antibody binds specifically to an epitope within a ABCG2 polypeptide. Non-specific binding would refer to binding with an affinity of less than about 10⁻⁷ M, e.g., binding with an affinity of 10⁻⁶ M, 10⁻⁵ M, 10⁻⁴ M, etc.

As used herein, the term “CDR” or “complementarity determining region” is intended to mean the non-contiguous antigen combining sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison.

TABLE 1

CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹ Residue numbering follows the nomenclature of Kabat et al., supra
² Residue numbering follows the nomenclature of Chothia et al., supra
³ Residue numbering follows the nomenclature of MacCallum et al., supra

As used herein, the term “framework” when used in reference to an antibody variable region is intended to mean all amino acid residues outside the CDR regions within the variable region of an antibody. A variable region framework is generally a discontinuous amino acid sequence between about 100-120 amino acids in length but is intended to reference only those amino acids outside of the CDRs. As used herein, the term “framework region” is intended to mean each domain of the framework that is separated by the CDRs. A VH chain can comprise three CDRs and four FRs arranged from N-terminus to C-terminus in the following order: FR1, CDR1, FR2, CDR2, FR3, CDR3, FR4. Similarly, a VL chain can comprise three CDRs and four FRs arranged from N-terminus to C-terminus in the following order: FR1, CDR1, FR2, CDR2, FR3, CDR3, FR4.

As used herein the term antibody encompasses a tetramer of two heavy and two light chains, wherein the heavy and light chains are interconnected by, for example, disulphide bonds. The heavy chain constant region is comprised of three domains, CH1, CH2 and CH3. The light chain constant region is comprised of one domain, CL. The variable regions of the heavy and light chains comprise binding regions that interact with antigen. The constant regions of the antibodies typically mediate the binding of the antibody to host tissues and factors, including various cells of the immune system and the first component of the complement system. The term “antibody” includes immunoglobulins of types IgA, IgG, IgE, IgD, IgM and subtypes thereof. In some embodiments, a subject antibody is an IgG isotype, e.g., IgG1.

As used herein the term “immunoglobulin” refers to a protein including one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes; and numerous immunoglobulin variable region genes. Full-length immunoglobulin light chains (about 25 kD or 214 amino acids) are encoded by a variable region gene at the N-terminus (about 110 amino acids) and a kappa or lambda constant region at the C-terminus. Full-length immunoglobulin heavy chains (about 50 kD or 446 amino acids) are encoded by a variable region gene at the N-terminus (about 116 amino acids) and one of the other aforementioned constant region genes at the C-terminus, e.g. gamma (encoding about 330 amino acids). In some embodiments, a subject antibody comprises a whole immunoglobulin comprising full-length immunoglobulin heavy chain and a full-length immunoglobulin light chain.

The term “antigen-binding fragment” refers to one or more fragments of a full-length antibody that are capable of specifically binding to an antigen. Examples of binding fragments include (i) a Fab fragment (a monovalent fragment including, e.g., consisting of, the VL, VH, CL and CH1 domains; (ii) a F(ab′)₂ fragment (a bivalent fragment comprising two Fab fragments linked by a disulfide bridge at the hinge region; (iii) a Fd fragment (including, e.g., consisting of, the VH and CH1 domains); (iv) a Fv fragment (including, e.g., consisting of, the VH and VL domains of a single arm of an antibody); (v) a dAb fragment (including, e.g., consisting of, the VH domain); (vi) an isolated CDR; (vii) a single chain Fv (scFv) (including, e.g., consisting of, the VH and VL domains of a single arm of an antibody joined by a synthetic linker using recombinant means such that the VH and VL domains pair to form a monovalent molecule); (viii) diabodies (including, e.g., consisting of, two scFvs in which the VH and VL domains are joined such that they do not pair to form a monovalent molecule; the VH of each one of the scFv pairs with the VL domain of the other scFv to form a bivalent molecule).

The term “chimeric” antibody refers to an antibody in which a portion of the heavy and/or light chain is derived from a particular source or species, while the remainder of the heavy and/or light chain is derived from a different source or species.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework (FR) which represents the most commonly occurring amino acid residues in a selection of human immunoglobulin variable light chain (VL) or variable heavy chain (VH) framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human CDRs and amino acid residues from human frameworks (FRs). At least a portion of a humanized antibody constant region derived from a human antibody. In preferred embodiments of the antibody molecules disclosed herein, the constant region is from a human IgG antibody, such as, human IgG1. In preferred embodiments, the antibody molecules disclosed herein include a heavy chain comprising a variable heavy chain region as provided herein and a human IgG1 constant region having the amino acid sequence sequence set forth in UniProt: P01857-1, version 1. In preferred embodiments, the antibody molecules disclosed herein include a light chain comprising a variable light chain region as provided herein and a human light chain constant region. In preferred embodiments, the human light chain constant region is a human kappa light chain constant region. In certain aspects, the human IgG1 heavy chain constant region present in the subject antibodies may include mutations, e.g., substitutions to modulate Fc function. For example, the LALAPG effector function mutations (L234A, L235A, and P329G) or the N297A mutation may be introduced to reduce antibody dependent cellular cytotoxicity (ADCC). The numbering of the substitutions is based on the EU numbering system. The “EU numbering system” or “EU index” is generally used when referring to a residue in an immunoglobulin heavy chain constant region (e.g., the EU index reported in Kabat et al., Sequences of Proteins of Immunological Interest, 5th Ed. Public Health Service, National Institutes of Health, Bethesda, MD. (1991)). The “EU index as in Kabat” refers to the residue numbering of the human IgG 1 EU antibody.

A “humanized form” of an antibody, e.g., a non-human antibody, refers to an antibody that has undergone humanization.

The term “epitope” refers to a region of an antigen that is recognized by the immune system, for example by antibodies, B cells, or T cells. For example, the epitope is the specific region of the antigen to which an antibody binds.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In some embodiments, the antibody will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated antibody includes the antibody in situ within recombinant cells since at least one component of the antibody’s natural environment will not be present. In some instances, isolated antibody will be prepared by at least one purification step.

The term “cytotoxic agent” as used herein refers to a substance that inhibits or prevents a cellular function and/or causes cell death or destruction. A “chemotherapeutic agent,” also referred to an “antineoplastic agent,” can be a cytotoxic agent which is used for treating a cancer or other disease or disorder.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, including in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” refers to the amount of a target-specific antibody that, when administered to a mammal or other subject for treating a disease, is sufficient to affect such treatment for the disease. The “therapeutically effective amount” will vary depending on the antibody, the disease and its severity and the age, weight, etc., of the subject to be treated.

The term “refractory”, used herein, refers to a disease or condition that does not respond to treatment. With regard to cancer, “refractory cancer”, as used herein, refers to cancer that does not respond to treatment. A refractory cancer may be resistant at the beginning of treatment or it may become resistant during treatment. Refractory cancer may also be called resistant cancer.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, serum, plasma, biological fluid, and tissue samples.

Percent identity between a pair of sequences may be calculated by multiplying the number of matches in the pair by 100 and dividing by the length of the aligned region, including gaps. Identity scoring only counts perfect matches and does not consider the degree of similarity of amino acids to one another. Only internal gaps are included in the length, not gaps at the sequence ends. Percent Identity = (Matches x 100)/Length of aligned region (with gaps)

The phrase “conservative amino acid substitution” refers to substitution of amino acid residues within the following groups: 1) L, I, M, V, F; 2) R, K; 3) F, Y, H, W, R; 4) G, A, T, S; 5) Q, N; and 6) D, E. Conservative amino acid substitutions may preserve the activity of the protein by replacing an amino acid(s) in the protein with an amino acid with a side chain of similar acidity, basicity, charge, polarity, or size of the side chain.

The term “vector” means any molecule or entity (e.g., nucleic acid, plasmid, bacteriophage or virus) used to transfer protein coding information into a host cell.

The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct may include, but is not limited to, sequences that affect or control transcription, translation, and, if introns are present, affect RNA splicing of a coding region operably linked thereto.

The term “stimulation,” refers to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules.

The term “stimulatory molecule,” refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In one aspect, the signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing cytoplasmic signaling sequence that is of particular use in the invention includes, but is not limited to, those derived from CD3 zeta, common FcR gamma (FCER1G), Fc gamma Rlla, FcR beta (Fc Epsilon R1b), CD3 gamma, CD3 delta, CD3 epsilon, CD79a, CD79b, DAP10, and DAP12.

The term a “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include, but are not limited to, an MHC class I molecule, BTLA and a Toll ligand receptor, as well as OX40, CD27, CD28, CDS, ICAM-1, LFA-1 (CD11a/CD18), ICOS (CD278), and 4-1BB (CD137).

The term “autologous” refers to any material derived from the same individual to whom it is later to be re-introduced into the individual.

An “intracellular signaling domain,” as the term is used herein, refers to an intracellular portion of a molecule. The intracellular signaling domain generates a signal that promotes an immune effector function of the CAR containing cell, e.g., a CAR-T cell. Examples of immune effector function, e.g., in a CAR-T cell, include cytolytic activity and helper activity, including the secretion of cytokines.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes.

Guidance for substitutions, insertions, or deletions may be based on alignments of amino acid sequences of proteins from different species or from a consensus sequence based on a plurality of proteins having the same or similar function.

### DETAILED DESCRIPTION

Provided are anti-ABCG2 antibodies that may be used as multi-specific antibodies that target both ABCG2 and a tumor-associated antigen (TAA) as well as pharmaceutical compositions, nucleic acids, recombinant expression vectors, cells, and kits that include or encode such multi-specific antibodies. The multi-specific antibodies include a common variable light (VL) chain that includes an antigen-binding site for ABCG2, a first variable heavy (VH) chain that includes an antigen-binding site for ABCG2, and a second VH chain that includes an antigen-binding site for the TAA or a first VH chain and a first VL chain that include an antigen-binding site for ABCG2 and a second VH chain and a second VL chain that include an antigen-binding site for the TAA, where the first and second VL chains are different. Methods of treating a subject for a cancer that include administering to the subject a multi-specific antibody that targets both ABCG2 and a TAA are also provided. The treating may involve administering the multi-specific antibody alone or administering the multi-specific antibody and a chemotherapeutic agent. Provided as well are methods of generating the described multi-specific antibodies and reagents related thereto, including genetically modified cell lines useful in the subject methods and methods of making such genetically modified cell lines.

The bispecific antibodies provided herein bind to cancer cells expressing both ABCG2 and the TAA while showing reduced binding to non-cancer cells expressing ABCG2 and/or the TAA. In other words, the bispecific antibodies provided herein bind with low affinity to (1) cells expressing TAA where ABCG2 expression is low or absent and (2) cells expressing ABCG2 where TAA expression is low or absent, and with high affinity to cancer cells that express at least one or both ABCG2 and TAA at relatively high levels, i.e., levels higher than normal cells.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the methods and compositions have or will be described for the sake of grammatical fluidity with functional explanations, it is to be expressly understood that the claims, unless expressly formulated under 35 U.S.C. §112(f), are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. §112(f) are to be accorded full statutory equivalents under 35 U.S.C. §112(f).

### Antibodies

### Bispecific Antibodies

The present disclosure provides a bispecific antibody molecule that binds multidrug resistance protein 1 (ABCG2) and a tumor associated antigen (TAA), the antibody molecule comprising two identical variable light (VL) chains, a first variable heavy (VH) chain, and a second VH chain, where the VL chains each comprise an antigen-binding site for ABCG2, the first VH chain comprises an antigen-binding site for ABCG2, and the second VH chain comprises an antigen-binding site for the TAA, and wherein the second VH chain binds the TAA when paired with one of the light chains or a first VH chain and a first VL chain that include an antigen-binding site for ABCG2 and a second VH chain and a second VL chain that include an antigen-binding site for a TAA, wherein the first and second VL chains have different sequences. The bispecific antibody molecule binds to cancer cells expressing both ABCG2 and the TAA while showing reduced binding to non-cancer cells expressing ABCG2 and/or the TAA. In other words, the bispecific antibodies provided herein bind with low affinity to (1) cells expressing TAA where ABCG2 expression is low or absent and (2) cells expressing ABCG2 where TAA expression is low or absent, and with high affinity to cancer cells that express at least one or both ABCG2 and TAA at relatively high levels, i.e., levels higher than normal cells.

Relatively high levels refer to expression that is at least 1.5 times (e.g., at least 2X, at least 3X, at least 4X, at least 5X or more) the expression level in a normal cell of the same type as the cancer cell. Reduced affinity refers to binding affinity that is reduced by at least 10% (e.g. at least 20%, at least 30%, at least 40%, at least 50% or more) as compared to binding of the antibody molecule to a normal cell of the same type as the cancer cell. Reduced affinity also encompasses lack of detectable binding.

The term “antibody molecule” encompasses antibodies as defined herein and includes antigen-binding fragments thereof. In certain aspects, the antibody molecule includes two variable light (VL) and two variable heavy (VH) chain. In certain aspects, the antibody molecule includes heavy chain and light chain constant regions as well. The heavy and light chain constant regions may be from a human antibody, e.g., human IgG1 antibody. The human IgG1 heavy chain (HC) constant region may be modified to include mutations that reduce antibody dependent cellular cytotoxicity (ADCC). In addition, or alternatively, the two VH chains may each be conjugated to a different human IgG1 HC constant region where the individual human IgG1 HC constant region has substitutions that favour formation of dimers between the different human IgG1 HC constant regions. Such HC regions are described in further detail herein. In certain aspects, where the antibody molecule is a bispecific antibody molecule, one of the human IgG1 HC constant regions may include substitutions to introduce one or more amino acids having a positively-charged side chain and the other human IgG1 HC constant region may include substitutions to introduce one or more amino acids having a negatively-charged side chain to favour formation of dimers between the two different HCs.

In certain aspects, the antigen binding sites of the two VL chains comprise the light chain CDRs (LCDRs1-3) of a VL chain having the sequence:

DIVLTQSPSSFSVSLGDRVTISCKASGYILNRLAWYQQKPGNAPRLLISG
ATSLETGFPSRFSGTGSGKDYTLSISSLQTEDVGTYYCQQYWSTPWTFGG
GTKLEIR (SEQ ID NO:1).

The VL chain amino acid sequence set forth in SEQ ID NO:1 is the sequence of VL chain of the anti-ABCG2 antibody 5D3.

In certain aspects, the CDRs of VL and VH light chains may be defined based on the Kabat nomenclature.

In certain aspects, i) the LCDR1 comprises the sequence: KASGYILNRLA (SEQ ID NO:2); (ii) the LCDR2 comprises the sequence: GATSLET (SEQ ID NO:3); and (iii) the LCDR3 comprises the sequence: QQYWSTPWT (SEQ ID NO:4). These LCDRs are based on the Kabat nomenclature.

In certain aspects, the two VL chains are humanized. In certain aspects, the two VL chains are humanized to include framework regions from a human antibody.

In certain aspects, the two VL chains comprise a sequence that is at least 80%, at least 85%, at least 90%, at least 95%, or a 100% identical to the sequence:

DIVLTQSPSSFSVSLGDRVTISCKASGYILNRLAWYQQKPGNAPRLLISG
ATSLETGFPSRFSGTGSGKDYTLSISSLQTEDVGTYYCQQYWSTPWTFGG
GTKLEIR (SEQ ID NO:1).

In certain aspects, the bispecific antibody includes a light chain comprising the VL chain as described herein and a light chain constant region. The light chain constant region may be the human immunoglobulin kappa chain constant region having the amino acid sequence set forth in UniProtKB/Swiss-Prot: P01834.2.

In certain aspects, the bispecific antibody molecule includes the first VH chain where the VH chain comprises heavy chain CDRs 1-3 (HCDRs 1-3) of a VH chain having the sequence:

QVQLQESGPGLVKPSQSLSLTCTVTGFSITSDYAWNWIRQFPGKKLEWMG
YINFDGGTTYNPSLRGRISITRDTSKNQFFLQLRSVTPEDTATYYCATFY
GAKGTLDYWGQGTSVTVSS (SEQ ID NO:5).

The VH chain amino acid sequence set forth in SEQ ID NO:5 is the sequence of VH chain of the anti-ABCG2 antibody 5D3.

In certain aspects, the HCDRs 1-3 of the VH chain are defined based on the Kabat nomenclature.

In certain aspects, the first VH chain comprises: (i) the HCDR1 comprising the sequence: SDYAWN (SEQ ID NO:63); (ii) the HCDR2 comprising the sequence: YINFDGGTTYNPSLRG (SEQ ID NO:64); and (iii) the HCDR3 comprising the sequence: FYGAKGTLDY (SEQ ID NO:65). These HCDRs are based on the Kabat nomenclature.

In certain aspects, the first and/or second VH chain is humanized. In certain aspects, the VH chains are humanized to include framework regions from a human antibody.

In certain aspects, the first VH chain comprises the sequence:

QVQLQESGPGLVKPSQSLSLTCTVTGFSITSDYAWNWIRQFPGKKLEWMG
YINFDGGTTYNPSLRGRISITRDTSKNQFFLQLRSVTPEDTATYYCATFY
GAKGTLDYWGQGTSVTVSS (SEQ ID NO:5);

EVQLQESGPGLVKPSETLSLTCTVSGFSITSDYAWNWIRQPPGKGLEWMG
YINFDGGTTYNPSLRGRITISRDTSKNQFSLKLSSVTAADTAVYYCATFY
GAKGTLDYWGQGTLVTVSS (SEQ ID NO:6); or

EVQLQESGPGLVKPSETLSLTCTVSGFSITSDYAWNWIRQPPGKGLEWIG
YINFDGGTTYNPSLRGRVTISRDTSKNQFSLKLSSVTAADTAVYYCATFY
GAKGTLDYWGQGTLVTVSS (SEQ ID NO:7).

In certain aspects, the first VH chain comprises a sequence that is at least 80%, at least 85%, at least 90%, or at least 95% identical to the sequence set forth in any one of SEQ ID NO:5, SEQ ID NO:6, or SEQ ID NO:7.

In certain aspects, the bispecific antibody includes a first heavy chain comprising the first VH chain as described herein and a human IgG1 heavy chain constant region.

In certain aspects, the second VH chain of the bispecific antibody is derived from a monospecific antibody molecule which binds the TAA, and wherein the affinity of the bispecific antibody molecule for the TAA is at least 2-fold lower (e.g. at least 3-fold lower, at least 4-fold lower, at least 5-fold lower) than the affinity of the monospecific antibody molecule for the TAA from which the VH chain is derived. The affinity of the bispecific antibody and the monospecific antibody is measured using the same assay. Any suitable method for measuring antibody affinity may be utilized. In certain aspects, affinity may be measured by calculating the equilibrium constant for the reversible binding of the antibody to an antigen and is expressed as a dissociation constant (Kd). In certain aspects, Kd may be measured by ELISA.

In certain aspects, the second VH chain of the bispecific antibody is derived from a monospecific antibody molecule which binds the TAA, and wherein the half-maximal effective concentration (EC50) of the bispecific antibody molecule for the TAA is at least 2-fold higher (e.g. at least 3-fold higher, at least 4-fold higher, at least 5-fold higher) than the EC50 of the monospecific antibody molecule for the TAA from which the VH chain is derived. The EC50 of the bispecific antibody and the monospecific antibody is measured using the same assay. Any suitable method for measuring EC50 of an antibody may be utilized. The concentration that provides half maximal response (e.g., half of the maximum fluorescence intensity) is measured as the EC50.

The EC50 of a test antibody many be determined by flow cytometry or ELISA. For example, flow cytometry may involve contacting a cell expressing an antigen (e.g. human wild type ABCG2 or a mutant ABCG2) with the antibody in a flow cytometry buffer, where the antibody is serially diluted, and incubating at room temperature or 4° C. for a period of time sufficient for the antibody to bind to the cells (e.g. 10 min-1 hr). After incubating, the cells may optionally be washed to remove and non-specifically bound antibody and/or the cells may be contacted with a fluorescently labeled secondary antibody that specifically binds to the test antibody. After incubation, the fluorescently labeled secondary antibody may be removed and the cells washed. The washed cells may be sorted by flow cytometry and the number of cells bound to the fluorescently labeled secondary antibody counted. The concentration that provides half maximal response (e.g., half of the maximum fluorescence intensity) is measured as the EC50. In variations of the flow cytometry assay, the cell may be a 293T cell overexpressing ABCG2.

The TAA may be any antigen that is known to be overexpressed in cancer cells. For example, the TAA may be an antigen that is not expressed at detectable levels in a normal cell and is expressed in cancer cells, where the normal and cancer cells are the same cell type, e.g., epithelial cells. For example, TAAs may be neoantigens that are a class of tumor antigens that arise from a tumor-specific mutation(s) which alters the amino acid sequence of encoded proteins as compared to the amino acid sequence of the unmutated protein. In other aspects, a TAA is an antigen that is expressed in normal cells but is expressed at higher levels in cancer cells. In certain aspects, the TAA may be CD47, ErbB1, ErbB2, or PD-L1.

### Anti-ABCG2 and Anti-CD47 Bispecific Antibody

In certain aspects, the bispecific antibody molecule binds to CD47 and the second VH chain comprises the HCDRs of a VH chain comprising the amino acid sequence:

QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSS (SEQ ID NO:8).

The VH chain amino acid sequence set forth in SEQ ID NO:8 is the sequence of VH chain of the anti-CD47 antibody 5F9.

In certain aspects, the second VH chain comprises the HCDR1 comprising the sequence: NYNMH (SEQ ID NO:9), the HCDR2 comprising the sequence: TIYPGNDDTSYNQKFKD (SEQ ID NO:10), and the HCDR3 comprising the sequence: GGYRAMDY (SEQ ID NO:11). The HCDRs1-3 are defined based on the Kabat nomenclature.

In certain aspects, the second VH chain comprises a sequence that is at least 80% identical (e.g., at least 85% identical, at least 90% identical, at least 95%, or 100% identical) to the amino acid sequence set forth in SEQ ID NO:8.

In certain aspects, the bispecific antibody comprises a first VH chain and a first VL chain and a second VH and a second VL chain where the first VH and VL chains bind to ABCG2 and the second VH and VL chains bind to CD47. The first VH and VL chains may be as described in the preceding section, where the common light chain is the first VL chain and the second VH chain is as described above and the second VL chain may be a VL chain comprising LCDRs1-3 of a VL chain of an anti-CD47 antibody. In certain aspects, the bispecific antibody comprises a first VH chain comprising HCDRs1-3 having the amino acid sequence set forth in SEQ ID Nos:63-65, respectively, and a first VL chain comprising LCDRs1-3 having the amino acid sequence set forth in SEQ ID Nos:2-4, respectively, and a second VH comprising HCDRs1-3 having the amino acid sequence set forth in SEQ ID Nos:9-11, respectively, and a second VL chain comprising LCDRs1-3 of the VL chain having the amino acid sequence set forth in SEQ ID No:60.

Additional aspects of the bispecific antibody are described elsewhere in the application and include humanized versions of the sequences disclosed herein and/or substitutions in the Fc region to promoter formation of heterodimers between the first and the second VH chains.

### Anti-ABCG2 and Anti-ErbB2 Bispecific Antibody

In certain aspects, the TAA may be ErbB2. ErbB2 is also known as Receptor Tyrosine Kinase 2 or HER2. In certain aspects, the bispecific antibody molecule that binds to ABCG2 and ErbB2 includes the common light chain and the first VH chain as described in the preceding sections and the second VH chain comprises the HCDRs 1-3 of a VH chain of mAb Pertuzumab comprising the amino acid sequence:

EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSS (SEQ ID NO:12).

The HCDRs 1-3 defined as per Kabat nomenclature are:

HCDR1: DYTMD (SEQ ID NO:13)

HCDR2: DVNPNSGGSIYNQRFKG (SEQ ID NO:14)

HCDR3: NLGPSFYFDY (SEQ ID NO:15)

The second VH chain of the bispecific antibody that binds to ABCG2 and ErbB2 may have an amino acid sequence at least 80%, at least 90%, at least 95%, or a 100% identical to the amino acid sequence set forth in SEQ ID NO:12.

The second VH chain of the bispecific antibody that binds to ABCG2 and ErbB2 may be present in heavy chain having an amino acid sequence at least 80%, at least 90%, at least 95%, or a 100% identical to the amino acid sequence:

EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRWSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG
(SEQ ID NO:16).

As described elsewhere herein, at least one, two, or all of the VL chain, the first VH chain, and the second VH may be humanized. In addition, the Fc regions of the VH chains may include substitutions to increase heterodimerization between the first and second VH chain. In certain aspects, the first heavy chain may be humanized and include the charged pair substitutions K392D and K409D and the second heavy chain and include the charged pair substitutions E356K and D399K.

In certain aspects, the bispecific antibody comprises a first VH chain and a first VL chain and a second VH and a second VL chain where the first VH and VL chains bind to ABCG2 and the second VH and VL chains bind to HER2. The first VH and VL chains may be as described in the preceding section, where the common light chain is the first VL chain and the second VH chain is as described above and the second VL chain may be a VL chain comprising LCDRs1-3 of a VL chain of an anti-HER2 antibody.

### Anti-ABCG2 and Anti-EGFR Bispecific Antibody

In certain aspects, the TAA may be epidermal growth factor receptor (EGFR). EGFR is also known as Receptor Tyrosine-Protein Kinase ErbB1 and HER1.The HCDRs1-3 for the second VH chain that includes an antigen-binding site for EGFR may be derived from the VH chain of the anti-EGFR antibody necitumumab or cetuximab. The heavy chain of necitumumab has the following sequence:

QVQLQESGPGLVKPSQTLSLTCTVSGGSISSGDYYWSWIRQPPGKGLEWI
GYIYYSGSTDYNPSLKSRVTMSVDTSKNQFSLKVNSVTAADTAVYYCARV
SIFGVGTFDYWGQGTLVTVSSASTKGPSVLPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
K (SEQ ID NO:17).

The heavy chain of cetuximab has the following sequence:

QVQLKQSGPGLVQPSQSLSITCTVSGFSLTNYGVHWVRQSPGKGLEWLGVI
WSGGNTDYNTPFTSRLSINKDNSKSQVFFKMNSLQSNDTAIYYCARALTYY
DYEFAYWGQGTLVTVSAASTKGPSVFPLAPSSKSTSGGTAALGCLVKDYFP
EPVTVSWNSGALTSGVHTFPAVLQSSGLYSLSSWTVPSSSLGTQTYICNVN
HKPSNTKVDKKVEPKSCDKTHTCPPCPAPELLGGPSVFLFPPKPKDTLMIS
RTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLY
SKLTVDKSRWQQGNVFSCSVMHEALHNHYTQKSLSLSPGK (SEQ ID NO
:18).

In a first aspect, the anti-ABCG2 anti-EGFR bispecific antibody includes the VL and first VH chain as described in the preceding sections and the second VH chain may include the HCDRs from VH region of necitumumab. The HCDRs defined as per Kabat nomenclature may have the following sequences:

HCDR1: SGDYYWS (SEQ ID NO:19)

HCDR2: YIYYSGSTDYNPSLKS (SEQ ID NO:20)

HCDR3: VSIFGVGTFDY (SEQ ID NO:21).

In certain aspects, the second VH chain may have an amino acid sequence at least 80%, at least 90%, at least 95%, or a 100% identical to the amino acid sequence:

QVQLQESGPGLVKPSQTLSLTCTVSGGSISSGDYYWSWIRQPPGKGLEWI
GYIYYSGSTDYNPSLKSRVTMSVDTSKNQFSLKVNSVTAADTAVYYCARV
SIFGVGTFDYWGQGTLVTVSS (SEQ ID NO:22).

The second VH chain of the bispecific antibody that binds to ABCG2 and EGFR may be present in heavy chain having an amino acid sequence at least 80%, at least 90%, at least 95%, or a 100% identical to the amino acid sequence set forth in SEQ ID NO:17.

In a second aspect, the anti-ABCG2 anti-EGFR bispecific antibody includes the VL and first VH chain as described in the preceding sections and the second VH chain may include the HCDRs from VH region of cetuximab. The HCDRs defined as per Kabat nomenclature may have the following sequences:

HCDR1: NYGVH (SEQ ID NO:23)

HCDR2: VIWSGGNTDYNTPFTS (SEQ ID NO:24)

HCDR3: ALTYYDYEFAY (SEQ ID NO:25)

In certain aspects, the second VH chain may have an amino acid sequence at least 80%, at least 90%, at least 95%, or a 100% identical to the amino acid sequence:

QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSA (SEQ ID NO:26)

The second VH chain of the bispecific antibody that binds to ABCG2 and EGFR may be present in heavy chain having an amino acid sequence at least 80%, at least 90%, at least 95%, or a 100% identical to the amino acid sequence set forth in SEQ ID NO: 18.

In certain aspects, the bispecific antibody comprises a first VH chain and a first VL chain and a second VH and a second VL chain where the first VH and VL chains bind to ABCG2 and the second VH and VL chains bind to EGFR. The first VH and VL chains may be as described in the preceding section, where the common light chain is the first VL chain and the second VH chain is as described above and the second VL chain may be a VL chain comprising LCDRs1-3 of a VL chain of an anti-EGFR antibody.

### Anti-ABCG2 and Anti-PD-L1 Bispecific Antibody

In certain aspects, the TAA may be Programmed death-ligand 1 (PD-L1). PD-L1 is also known as cluster of differentiation 274 (CD274) or B7 homolog 1 (B7-H1). In certain aspects, the bispecific antibody molecule that binds to ABCG2 and PD-L1 includes the common light chain and the first VH chain as described in the preceding sections and the second VH chain comprises the HCDRs 1-3 of a VH chain comprising the amino acid sequence:

EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS (SEQ ID NO:27)

The HCDRs 1-3 defined as per Kabat nomenclature are:

HCDR1: DSWIH (SEQ ID NO:28)

HCDR2: WISPYGGSTYYADSVKG (SEQ ID NO:29)

HCDR3: RHWPGGFDY (SEQ ID NO:30)

The second VH chain of the bispecific antibody that binds to ABCG2 and PD-L1 may have an amino acid sequence at least 80%, at least 90%, at least 95%, or a 100% identical to the amino acid sequence set forth in SEQ ID NO:27.

The second VH chain of the bispecific antibody that binds to ABCG2 and PD-L1 may be present in heavy chain having an amino acid sequence at least 80%, at least 90%, at least 95%, or a 100% identical to the amino acid sequence:

EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
(SEQ ID NO:31)

In certain aspects, the bispecific antibody comprises a first VH chain and a first VL chain and a second VH and a second VL chain where the first VH and VL chains bind to ABCG2 and the second VH and VL chains bind to PD-L1. The first VH and VL chains may be as described in the preceding section, where the common light chain is the first VL chain and the second VH chain is as described above and the second VL chain may be a VL chain comprising LCDRs1-3 of a VL chain of an anti-PD-L1 antibody.

As described elsewhere herein, at least one, two, or all of the VL chain (or the first and second VL chains), the first VH chain, and the second VH may be humanized. In addition, the Fc regions of the VH chains may include substitutions to increase heterodimerization between the first and second VH chain. In certain aspects, the first heavy chain may be humanized.

The first HC may include the charged pair substitutions K392D and K409D and the second heavy chain may include the charged pair substitutions E356K and D399K or vice versa.

In certain aspects, the bispecific antibody includes a second heavy chain comprising the second VH chain as described herein and a heavy chain constant region. The heavy chain may comprise the human IgG1 heavy chain constant region sequence.

In certain aspects, the bispecific antibody molecule specifically binds a cell expressing both ABCG2 and the TAA and has greater than twice the affinity for a cell expressing both ABCG2 and the TAA as compared to a cell expressing either ABCG2 or the TAA.

In certain aspects, the bispecific antibody molecule when bound to a cell expressing a ABCG2, inhibits efflux by the ABCG2. Inhibition may be a decrease in efflux by at least 5%, at least 10%, at least 15%, at least 20%, at least 25%, at least 50%, or more, as compared to efflux by the ABCG2 in absence of the bispecific antibody.

In certain aspects, the bispecific antibody molecule comprises a Fc domain that has been modified to reduce or abrogate binding of the antibody to one or more Fcy receptors. In certain aspects, the IgG1 Fc domain may have one or more of the substitutions L234A, L235A, P329G and N297A/Q/G.

As summarized above, the present disclosure provides multi-specific antibodies having a domain that targets a cellular efflux pump and a domain that targets a cancer-associated antigen. Included are multi-specific antibodies that include a multidrug resistance protein 1 (ABCG2)-binding domain and a leukocyte surface antigen CD47-binding domain. Such multi-specific antibodies of the present disclosure specifically bind cells that express both ABCG2 and CD47.

In one aspect, the multi-specific antibodies of the present disclosure target both ABCG2 and CD47. ABCG2, also known as CD388 and BCRP, is an energy-dependent efflux pump responsible for decreased drug accumulation in multidrug-resistant cells that is expressed from the ATP binding cassette subfamily G member 2 (ABCG2) gene. CD47, also known as integrin associated protein (IAP), is an immunoglobulin superfamily transmembrane protein that binds membrane integrins and also serves as a receptor for the ligands thrombospondin-1 (TSP-1) and signal-regulatory protein alpha (SIRPα) and is encoded by the CD47 gene. CD47 ligand binding can result in inhibition of phagocytosis and thus, as a target in immune therapy, masking of the CD47 extracellular domain prevents inhibition of immune-mediated killing of CD47-expressing cancer cells.

A multi-specific antibody of the present disclosure includes a ABCG2-binding domain and a CD47-binding domain and, optionally, all or a portion of an Fc domain. In the presence of a cell that expresses ABCG2, but where CD47 expression is low or absent, the multi-specific antibody has low affinity for the cell. Correspondingly, in the presence of a cell that expresses CD47, but where ABCG2 expression is low or absent, the multi-specific antibody has low affinity for the cell. However, in the presence of a cell that expresses both ABCG2 and CD47, the multi-specific antibody has high affinity for the cell.

Thus, multi-specific antibodies of the present disclosure bind cells that express both ABCG2 and CD47 with higher affinity than cells that express only ABCG2 or CD47. Correspondingly, multi-specific antibodies of the present disclosure bind with much reduced affinity when low levels of the respective second target are present, e.g., as compared to when both first and second targets are present above low levels (e.g., at average, normal, and/or high levels). In some embodiments, the affinity with which the subject multi-specific antibodies bind cells that express both ABCG2 and CD47 is greater than twice, including e.g., greater than 2.5 times, greater than 3 times, greater than 4 times, greater than 5 times, greater than 6 times, greater than 7 times, greater than 8 times, greater than 9 times, greater than 10 times, or more, as compared to the affinity with which the subject multi-specific antibodies bind cells that express either ABCG2 or CD47 (or a low level of either ABCG2 or CD47).

In some embodiments, the subject multi-specific antibodies may, when bound to a cell expressing ABCG2, prevent the functioning of the cellular ABCG2 protein. Accordingly, multi-specific antibodies of the present disclosure may inhibit efflux by the ABCG2 protein, including e.g., where efflux is reduced by 5% or more, including e.g., 10% or more, 15% or more, 20% or more, 25% or more, 30% or more, 40% or more, 50% or more, 60% or more, 70% or more, 80% or more, or 90% or more, as compared to efflux by ABCG2 in the absence of the subject multi-specific antibody.

In some embodiments, the subject multi-specific antibodies may, when bound to a cell expressing CD47, prevent the functioning of the cellular CD47 protein. Accordingly, multi-specific antibodies of the present disclosure may inhibit binding of a CD47-ligand or CD47 binding partner to CD47, including e.g., where ligand/binding partner binding is reduced by 5% or more, including e.g., 10% or more, 15% or more, 20% or more, 25% or more, 30% or more, 40% or more, 50% or more, 60% or more, 70% or more, 80% or more, or 90% or more, as compared to binding by CD47 in the absence of the subject multi-specific antibody.

Multi-specific antibodies of the present disclosure are at least bispecific for ABCG2 and CD47, where the configuration of the antibody may vary. The term “antibody” refers to a protein comprising one or more (e.g., one or two) heavy chain variable regions (VH) and/or one or more (e.g., one or two) light chain variable regions (VL), or subfragments thereof capable of binding an epitope. With regard to the herein described multi-specific antibodies, such antibodies are capable of binding at least two different epitopes present on two different target proteins. The number of different target proteins, and thus different epitopes, bound by the subject multi-specific antibodies may vary and may be two (i.e., bispecific), three (tri-specific), four, or greater.

In some embodiments, multi-specific antibodies of the present disclosure may include a common light chain. As used herein, the term “common light chain” will generally refer to the use, and incorporation, of two copies of the same light chain into the multi-specific antibody. Put another way, a light chain, in the assembled multi-specific antibody, will associate with the ABCG2-specific heavy chain and a second copy of the same light chain will associate with the CD47-specific heavy chain.

The VH and VL regions can be further subdivided into regions of hypervariability, termed “complementarity determining regions (CDR)”, interspersed with regions that are more conserved, termed “framework regions (FR)”. The extent of the FR and CDRs has been precisely defined (see, Kabat, et al. (1991) Sequences of Proteins of Immunological Interest, Fifth Edition, U.S. Department of Health and Human Services, NIH Publication No. 91-3242; Chothia et al. (1987) J. Mol. Biol. 196: 901-917). A VH can comprise three CDRs and four FRs arranged from N-terminus to C-terminus in the following order: FR1, CDR1, FR2, CDR2, FR3, CDR3, FR4. Similarly, a VL can comprise three CDRs and four FRs arranged from N-terminus to C-terminus in the following order: FR1, CDR1, FR2, CDR2, FR3, CDR3, FR4.

The VH or VL chain of an antibody can further include all or part of a heavy or light chain constant region, to thereby form a heavy or light immunoglobulin chain, respectively. In one embodiment, the antibody is a tetramer of two heavy and two light chains, wherein the heavy and light chains are interconnected by, for example, disulphide bonds. The heavy chain constant region is comprised of three domains, CH1, CH2 and CH3. The light chain constant region is comprised of one domain, CL. The variable regions of the heavy and light chains comprise binding regions that interact with antigen. The constant regions of the antibodies typically mediate the binding of the antibody to host tissues and factors, including various cells of the immune system and the first component of the complement system. The term “antibody” includes immunoglobulins of types IgA, IgG, IgE, IgD, IgM and subtypes thereof. In some embodiments, a subject antibody is an IgG isotype.

As used herein the term “immunoglobulin” refers to a protein including one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes; and numerous immunoglobulin variable region genes. Full-length immunoglobulin light chains (about 25 kD or 214 amino acids) are encoded by a variable region gene at the N-terminus (about 110 amino acids) and a kappa or lambda constant region at the C-terminus. Full-length immunoglobulin heavy chains (about 50 kD or 446 amino acids) are encoded by a variable region gene at the N-terminus (about 116 amino acids) and one of the other aforementioned constant region genes at the C-terminus, e.g. gamma (encoding about 330 amino acids). In some embodiments, a subject antibody comprises full-length immunoglobulin heavy chain and a full-length immunoglobulin light chain.

In some embodiments, a subject antibody does not comprise a full-length immunoglobulin heavy chain and a full-length immunoglobulin light chain, and instead comprises antigen-binding fragments of one or more full-length immunoglobulin heavy chains and/or one or more antigen-binding fragments of a full-length immunoglobulin light chain. In some embodiments, the antigen-binding fragments are contained on separate polypeptide chains; in other embodiments, the antigen-binding fragments are contained within a single polypeptide chain.

The term “antigen-binding fragment” refers to one or more fragments of a full-length antibody that are capable of specifically binding to ABCG2 or CD47 as described above. Examples of binding fragments include (i) a Fab fragment (a monovalent fragment including, e.g., consisting of, the VL, VH, CL and CH1 domains; (ii) a F(ab′)₂ fragment (a bivalent fragment comprising two Fab fragments linked by a disulfide bridge at the hinge region; (iii) a Fd fragment (including, e.g., consisting of, the VH and CH1 domains); (iv) a Fv fragment (including, e.g., consisting of, the VH and VL domains of a single arm of an antibody); (v) a dAb fragment (including, e.g., consisting of, the VH domain); (vi) an isolated CDR; (vii) a single chain Fv (scFv) (including, e.g., consisting of, the VH and VL domains of a single arm of an antibody joined by a synthetic linker using recombinant means such that the VH and VL domains pair to form a monovalent molecule); (viii) diabodies (including, e.g., consisting of, two scFvs in which the VH and VL domains are joined such that they do not pair to form a monovalent molecule; the VH of each one of the scFv pairs with the VL domain of the other scFv to form a bivalent molecule).

In some embodiments, a subject antibody is a recombinant or modified antibody, e.g., a chimeric, humanized, deimmunized or an in vitro generated antibody. The term “recombinant” or “modified” antibody as used herein is intended to include all antibodies that are prepared, expressed, created, or isolated by recombinant means, such as (i) antibodies expressed using a recombinant expression vector transfected into a host cell; (ii) antibodies isolated from a recombinant, combinatorial antibody library; (iii) antibodies isolated from an animal (e.g. a mouse) that is transgenic for human immunoglobulin genes; or (iv) antibodies prepared, expressed, created, or isolated by any other means that involves splicing of human immunoglobulin gene sequences to other DNA sequences. Such recombinant antibodies include humanized, CDR grafted, chimeric, deimmunized, and in vitro generated antibodies; and can optionally include constant regions derived from human germline immunoglobulin sequences.

Modified antibodies may include modified domains, including where any antibody domain may be modified from a naturally occurring form. In some embodiments, a modified antibody may include a modified heavy chain, including a modified Fc domain, including a modified CH2 and/or modified CH3 domain. In some instances, modified Fc domains may employ electrostatic steering effects, including but not limited to e.g., through the use of the procedures described in Gunasekeran et al, (2010) Journal of Biological Chemistry 285, 19637-19646; the disclosure of which is incorporated herein by reference in its entirety. In some instances, a bispecific antibody is assembled through charge pair substitutions at the CH3 domain, including but not limited to e.g., where one heavy chain is modified to contain K392D and K409D substitutions and the other heavy chain is modified to contained E356K and D399K substitutions. Charge pair substituted chains may preferentially form a heterodimer with one another. The numbering of the amino acid substitutions is per EU numbering system for HCs.

In some instances, an antibody of the present disclosure includes charge pair substitutions. In some instances, an antibody of the present disclosure does not include charge pair substitutions. In some instances, an alternative means of promoting preferential heterodimer formation of desired chains may be employed.

In some instances, a modified heavy chain may include a knob-into-hole modification. “Knobs-into-holes” amino acid modification is a rational design strategy in antibody engineering, used for heterodimerization of the heavy chains, in the production of multi-specific antibodies, including bispecific IgG antibodies. For example, in incorporating the knobs-into-holes strategy into a bispecific antibody made from two monoclonal antibodies of different specificities, amino acid changes are engineered in order to create a “knob” on the CH3 of the heavy chain of monoclonal antibody 1 (mAb1) and a “hole” on the CH3 of the heavy chain of monoclonal antibody 2 (mAb2). The knob may be represented by a large amino acid, such as e.g., a tyrosine (Y), whereas the hole may be represented by small amino acid, such as a threonine (T). For example, a knobs-into-holes pair modification may be created a T22Y substitution in a first CH3 domain and Y86T substitution in the partner CH3 domain. Examples of knobs-into-holes modifications are described in Carter, J. Immunol. Methods, 248(1-2):7-15 (2001); Ridgway, J. B. et al. Protein Eng. 9(7):617-2 (1996); and Merchant, A. M. et al. Nat. Biotechnol. 16(7):677-81 (1998); the disclosures of which are incorporated herein in their entirety. In antibodies generated from paired knob-into-hole modified domains the bispecific heterodimer will generally represent the major fraction.

As summarized above, multi-specific antibodies of the present disclosure will include a ABCG2-binding domain and CD47-binding domain. Such domains may vary, including the epitopes bound by the domains, the variable region arrangement and sequences, etc.

A subject ABCG2-binding domain specifically binds one or more epitopes of ABCG2. Thus, the epitope is a ABCG2 epitope. The size of a ABCG2 epitope bound by a ABCG2-binding domain may vary, including where the ABCG2 epitope is formed by a polypeptide having a contiguous stretch of a ABCG2 sequence that may range from 4 aa or less to 12 aa or more, including but not limited to e.g., 4 aa, 5 aa, 6 aa, 7 aa, 8 aa, 9 aa, 10 aa, 11 aa, 12 aa, 4 aa to 10 aa, 5 aa to 10 aa, 6 aa to 10 aa, 4 aa to 8 aa, 5 aa to 8 aa, 6 aa to 8 aa, etc.

In some embodiments, the ABCG2 epitope can be formed by a polypeptide having at least about 75%, at least about 80%, at least about 85%, at least about 90%, at least about 95%, at least about 98%, at least about 99%, or 100%, amino acid sequence identity to a contiguous stretch of a ABCG2 sequence, including but not limited to e.g., the human ABCG2 sequence:

MSSSNVEVFIPVSQGNTNGFPATASNDLKAFTEGAVLSFHNICYRVKLKS
GFLPCRKPVEKEILSNINGIMKPGLNAILGPTGGGKSSLLDVLAARKDPS
GLSGDVLINGAPRPANFKCNSGYVVQDDVVMGTLTVRENLQFSAALRLAT
TMTNHEKNERINRVIQELGLDKVADSKVGTQFIRGVSGGERKRTSIGMEL
ITDPSILFLDEPTTGLDSSTANAVLLLLKRMSKQGRTIIFSIHQPRYSIF
KLFDSLTLLASGRLMFHGPAQEALGYFESAGYHCEAYNNPADFFLDIING
DSTAVALNREEDFKATEIIEPSKQDKPLIEKLAEIYVNSSFYKETKAELH
QLSGGEKKKKITVFKEISYTTSFCHQLRWVSKRSFKNLLGNPQASIAQII
VTVVLGLVIGAIYFGLKNDSTGIQNRAGVLFFLTTNQCFSSVSAVELFVV
EKKLFIHEYISGYYRVSSYFLGKLLSDLLPMRMLPSIIFTCIVYFMLGLK
PKADAFFVMMFTLMMVAYSASSMALAIAAGQSVVSVATLLMTICFVFMMI
FSGLLVNLTTIASWLSWLQYFSIPRYGFTALQHNEFLGQNFCPGLNATGN
NPCNYATCTGEEYLVKQGIDLSPWGLWKNHVALACMIVIFLTIAYLKLLF
LKKYS (SEQ ID NO:32),

or the ECD1 (417 - 428) thereof:

KNDSTGIQNRAG (SEQ ID NO:33);

the ECD2 (499 - 506) thereof:

LKPKADAF(SEQ ID NO:34);

the ECD3 (557 - 630) thereof:

NLTTIASWLSWLQYFSIPRYGFTALQHNEFLGQNFCPGLNATGNNPCNY
ATCTGEEYLVKQGIDLSPWGLWKNH (SEQ ID NO:35);

or a Mus musculus ABCG2 sequence:

MSSSNDHVLVPMSQRNNNGLPRTNSRAVRTLAEGDVLSFHHITYRVKVK
SGFLVRKTVEKEILSDINGIMKPGLNAILGPTGGGKSSLLDVLAARKDP
KGLSGDVLINGAPQPAHFKCCSGYVVQDDVVMGTLTVRENLQFSAALRL
PTTMKNHEKNERINTIIKELGLEKVADSKVGTQFIRGISGGERKRTSIG
MELITDPSILFLDEPTTGLDSSTANAVLLLLKRMSKQGRTIIFSIHQPR
YSIFKLFDSLTLLASGKLVFHGPAQKALEYFASAGYHCEPYNNPADFFL
DVINGDSSAVMLNREEQDNEANKTEEPSKGEKPVIENLSEFYINSAIYG
ETKAELDQLPGAQEKKGTSAFKEPVYVTSFCHQLRWIARRSFKNLLGNP
QASVAQLIVTVILGLIIGAIYFDLKYDAAGMQNRAGVLFFLTTNQCFSS
VSAVELFVVEKKLFIHEYISGYYRVSSYFFGKVMSDLLPMRFLPSVIFT
CVLYFMLGLKKTVDAFFIMMFTLIMVAYTASSMALAIATGQSWSVATLL
MTIAFVFMMLFSGLLVNLRTIGPWLSWLQYFSIPRYGFTALQYNEFLGQ
EFCPGFNVTDNSTCVNSYAICTGNEYLINQGIELSPWGLWKNHVALACM
IIIFLTIAYLKLLFLKKYS (SEQ IDNO:36),

or the ECD1 (415 - 428) thereof:

DLKYDAAGMQNRAG (SEQ ID NO:37);

the ECD2 (499 - 506) thereof:

LKKTVDAF (SEQ ID NO:38);

the ECD3 (557 - 632) thereof:

NLRTIGPWLSWLQYFSIPRYGFTALQYNEFLGQEFCPGFNVTDNSTCVNS
YAICTGNEYLINQGIELSPWGLWKNH (SEQ ID NO:39);

a non-human primate sequence, such as e.g., the Macaca fascicularis (Crab-eating macaque) sequence:

MSSSNVEVFIPMSQENTNGFPTTTSNDRKAFTEGAVLSFHNICYRVKVKS
GFLPGRKPVEKEILSNINGIMKPGLNAILGPTGGGKSSLLDVLAARKDPS
GLSGDVLINGALRPTNFKCNSGYVVQDDVVMGTLTVRENLQFSAALRLPT
TMTNHEKNERINRVIQELGLDKVADSKVGTQFIRGVSGGERKRTSIGMEL
ITDPSILFLDEPTTGLDSSTANAVLLLLKRMSKQGRTIIFSIHQPRYSIF
KLFDSLTLLASGRLMFHGPAQEALGYFESAGYHCEAYNNPADFFLDIING
DSTAVALNREEDFKATEIIEPSKRDKPLVEKLAEIYVDSSFYKETKAELH
QLSGGEKKKKITVFKEISYTTSFCHQLRWVSKRSFKNLLGNPQASIAQII
VTVILGLVIGAIYFGLNNDSTGIQNRAGVLFFLTTNQCFSSVSAVELFVV
EKKLFIHEYISGYYRVSSYFFGKLLSDLLPMRMLPSIIFTCIVYFMLGLK
PTADAFFIMMFTLMMVAYSASSMALAIAAGQSVVSVATLLMTICFVFMMI
FSGLLVNLTTIASWLSWLQYFSIPRYGFTALQHNEFLGQNFCPGLNATVN
NTCNYATCTGEEYLTKQGIDLSPWGLWKNHVALACMIVIFLTIAYLKLLF
LKKYS (SEQ ID NO:40),

or the ECD1 (417 - 428) thereof:

NNDSTGIQNRAG (SEQ ID NO:41);

the ECD2 (499 - 506) thereof:

LKPTADAF (SEQ ID NO:42);

the ECD3 (557 - 630) thereof:

NLTTIASWLSWLQYFSIPRYGFTALQHNEFLGQNFCPGLNATVNNTCNYA
TCTGEEYLTKQGIDLSPWGLWKNH (SEQ ID NO:43),

or Pan troglodytes ABCG2 sequence:

MSSSNVEVFIPMSQGNTNGFPATTSNDLKAFTEGAVLSFHNICYRVKLKS
GFLPCRKPVEKEILSNINGIMKPGLNAILGPTGGGKSSLLDVLAARKDPS
GLSGDVLINGAPRPANFKCNSGYVVQDDVVMGTLTVRENLQFSAALRLPT
TMTNHEKNERINRVIQELGLDKVADSKVGTQFIRGVSGGERKRTSIGMEL
ITDPSILFLDEPTTGLDSSTANAVLLLLKRMSKQGRTIIFSIHQPRYSIF
KLFDSLTLLASGRLMFHGPAQEALGYFESAGYHCEAYNNPADFFLDIING
DSTAVALNREEDFKATEIIEPSKQDKPLIEKLAEIYVNSSFYKETKAELH
QLSGGEKKKKITVFKEISYTTSFCHQLRWVSKRSFKNLLGNPQASIAQII
VTVILGLVIGAIYFGLKNDSTGIQNRAGVLFFLTTNQCFSSVSAVELFVV
EKKLFIHEYISGYYRVSSYFLGKLLSDLLPMRMLPSIIFTCIVYFMLGLK
PKADAFFVMMFTLMMVAYSASSMALAIAAGQSWSVATLLMTICFVFMMIF
SGLLVNLTTIASWLSWLQYFSIPRYGFTALQHNEFLGQNFCPGLNATGNN
PCNYATCTGEEYLVKQGIDLSPWGLWKNHVALACMIVIFLTIAYLKLLFL
KKYS (SEQ ID NO:44),

or the like.

In some embodiments, the ABCG2 epitope can be formed by a mutated ABCG2 polypeptide. The mutated ABCG2 polypeptide may be derived from a human ABCG2 polypeptide. The human ABCG2 polypeptide may include a mutation that results in the ABCG2 polypeptide having an open configuration. A mutant human ABCG2 polypeptide having an open configuration may include the substitution: E211Q, numbered with reference to the sequence of human ABCG2 polypeptide as provided herein. In certain aspects, a mutant human ABCG2 polypeptide having an open configuration may comprise an amino acid sequence that is at least 80% (e.g. at least 85%, at least about 90%, at least about 95%, at least about 98%, at least about 99%, or 100%) identical to the amino acid sequence:

MSSSNVEVFIPVSQGNTNGFPATASNDLKAFTEGAVLSFHNICYRVKLKS
GFLPCRKPVEKEILSNINGIMKPGLNAILGPTGGGKSSLLDVLAARKDPS
GLSGDVLINGAPRPANFKCNSGYWQDDVVMGTLTVRENLQFSAALRLATT
MTNHEKNERINRVIQELGLDKVADSKVGTQFIRGVSGGERKRTSIGMELI
TDPSILFLDQPTTGLDSSTANAVLLLLKRMSKQGRTIIFSIHQPRYSIFK
LFDSLTLLASGRLMFHGPAQEALGYFESAGYHCEAYNNPADFFLDIINGD
STAVALNREEDFKATEIIEPSKQDKPLIEKLAEIYVNSSFYKETKAELHQ
LSGGEKKKKITVFKEISYTTSFCHQLRWVSKRSFKNLLGNPQASIAQIIV
TVVLGLVIGAIYFGLKN DSTGIQNRAGVLFFLTTNQCFSSVSAVELFVV
EKKLFIHEYSGYYRVSSYFLGKLLSDLLPMRMLPSIIFTCIVYFMLGLKP
KADAFFVMMFTLMMVAYSASSMALAIAAGQSVVSVATLLMTICFVFMMIF
SGLLVNLTTIASWLSWLQYFSIPRYGFTALQHNEFLGQNFCPGLNATGNN
PCNYATCTGEEYLVKQGIDLSPWGLWKNHVALACMIVIFLTIAYLKLLFL
KKYS (SEQ ID NO:45),

or the like.

A subject ABCG2-binding domain exhibits high affinity binding to ABCG2. For example, a subject ABCG2-binding domain binds to ABCG2 with an affinity of at least about 10⁻⁷ M, at least about 10⁻⁸ M, at least about 10⁻⁹ M, at least about 10⁻¹⁰ M, at least about 10⁻¹¹ M, or at least about 1 y, or greater than 10⁻¹² M. A subject ABCG2-binding domain binds to an epitope present on ABCG2with an affinity of from about 10⁻⁷ M to about 10⁻⁸ M, from about 10⁻⁸ M to about 10⁻⁹ M, from about 10⁻⁹ M to about 10⁻¹⁰ M, from about 10⁻¹⁰ M to about 10⁻¹¹ M, or from about 10⁻¹¹ M to about 10⁻¹² M, or greater than 10⁻¹² M.

A subject ABCG2-binding domain exhibits substantially no binding to any epitopes formed by amino acids within other related, but sequence dissimilar, proteins such as related but sequence dissimilar EPs. Any binding of a subject ABCG2-binding domain to an epitope formed by amino acids within a related, but sequence dissimilar, protein is generally non-specific binding of a substantially lower affinity than the specific binding of the ABCG2-binding domain to the epitope on ABCG2. A substantially lower affinity is generally at least a two fold, three fold, five fold, 10 fold, 50 fold, 100 fold, 500 fold, or 1000 fold lower affinity.

A subject ABCG2-binding domain can reduce transport of molecules through a ABCG2 transporter. For example, a subject ABCG2-binding domain can reduce transport by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the degree of transport in the absence of the ABCG2-binding domain.

A subject CD47-binding domain specifically binds one or more epitopes of CD47. Thus, the epitope is a CD47 epitope. The size of a CD47 epitope bound by a CD47-binding domain may vary, including where the CD47 epitope is formed by a polypeptide having a contiguous stretch of a CD47 sequence that may range from 4 aa or less to 12 aa or more, including but not limited to e.g., 4 aa, 5 aa, 6 aa, 7 aa, 8 aa, 9 aa, 10 aa, 11 aa, 12 aa, 4 aa to 10 aa, 5 aa to 10 aa, 6 aa to 10 aa, 4 aa to 8 aa, 5 aa to 8 aa, 6 aa to 8 aa, etc.

In some embodiments, the CD47 epitope can be formed by a polypeptide having at least about 75%, at least about 80%, at least about 85%, at least about 90%, at least about 95%, at least about 98%, at least about 99%, or 100%, amino acid sequence identity to a contiguous stretch of a CD47 sequence, including but not limited to e.g., the human CD47 sequence:

MWPLVAALLLGSACCGSAQLLFNKTKSVEFTFCNDTVVIPCFVTNMEAQNT
TEVYVKWKFKGRDIYTFDGALNKSTVPTDFSSAKIEVSQLLKGDASLKMDK
SDAVSHTGNYTCEVTELTREGETIIELKYRWSWFSPNENILIVIFPIFAIL
LFWGQFGIKTLKYRSGGMDEKTIALLVAGLVITVIVIVGAILFVPGEYSLK
NATGLGLIVTSTGILILLHYYVFSTAIGLTSFVIAILVIQVIAYILAVVGL
SLCIAACIPMHGPLLISGLSILALAQLLGLVYMKFVASNQKTIQPPRKAVE
EPLNAFKESKGMMNDE (SEQ ID NO:46);

or a rodent CD47 sequence, such as e.g., the mouse CD47 sequence:

MWPLAAALLLGSCCCGSAQLLFSNVNSIEFTSCNETVVIPCIVRNVEAQS
TEEMFVKWKLNKSYIFIYDGNKNSTTTDQNFTSAKISVSDLINGIASLKM
DKRDAMVGNYTCEVTELSREGKTVIELKNRTVSWFSPNEKILIVIFPILA
ILLFWGKFGILTLKYKSSHTNKRIILLLVAGLVLTVIWVGAILLIPGEKP
VKNASGLGLIVISTGILILLQYNVFMTAFGMTSFTIAILITQVLGYVLAL
VGLCLCIMACEPVHGPLLISGLGIIALAELLGLVYMKFVASNQRTIQPPR
NR (SEQ ID NO:47),

or a non-human primate sequence, such as e.g., the Pongo abelii (Sumatran orangutan) sequence:

MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELTREGETIIELKYRWSWFSPNENILIVIFPIF
AILLFWGQFGIKTLKYRSGGMDEKTIALLVAGLIITVIVIVGAILFVPGE
YSLKNATGLGLIVTSTGILILLHYYVFSTAIGLNSFVIAILVIQVIAYIL
AWGLSLCIAACIPMHGPLLISGLSILALAQLLGLVYMKFVASNQKTIQPP
RKAVEEPLNAFKESKGMMNDE (SEQ ID NO:48),

or the Macaca mulatta (Rhesus macaque) sequence:

MWPLVAALLLGSACCGSAQLLFNKTKSVEFTFCNDTVVIPCFVTNMEAQN
TTEVYVKWKFKGRDIYTFDGALNKSTAPANFSSAKIEVSQLLKGDASLKM
DKSDAVSHTGNYTCEVTELTREGETIIELKYRWSWFSPNENILIVIFPIF
AILLFWGQFGIKTLKYRSGGMDEKTIALLVAGLMITVIVIVGAILFVPGE
YSLKNATGLGLIVTSTGILILLHYYVFSTAIGLTSFVIAILVIQVIAYIL
AWGLSLCIAACIPMHGPLLISGLSILALAQLLGLVYMKFVASNQKTIQPP
RKAVEEPLNAFKESKGMMNDE (SEQ ID NO:49),

or the like.

A subject CD47-binding domain exhibits high affinity binding to CD47. For example, a subject CD47-binding domain binds to CD47 with an affinity of at least about 10⁻⁷ M, at least about 10⁻⁸ M, at least about 10⁻⁹ M, at least about 10⁻¹⁰ M, at least about 10⁻¹¹ M, or at least about 10⁻¹² M, or greater than 10⁻¹² M. A subject CD47-binding domain binds to an epitope present on CD47 with an affinity of from about 10⁻⁷ M to about 10⁻⁸ M, from about 10⁻⁸ M to about 10⁻⁹ M, from about 10⁻⁹ M to about 10⁻¹⁰ M, from about 10⁻¹⁰ M to about 10⁻¹¹ M, or from about 10⁻¹¹ M to about 10⁻¹² M, or greater than 10⁻¹² M.

A subject CD47-binding domain exhibits substantially no binding to any epitopes formed by amino acids within other related, but sequence dissimilar, proteins such as related but sequence dissimilar immune checkpoint markers. Any binding of a subject CD47-binding domain to an epitope formed by amino acids within a related, but sequence dissimilar, protein is generally non-specific binding of a substantially lower affinity than the specific binding of the CD47-binding domain to the epitope on CD47. A substantially lower affinity is generally at least a twofold, three fold, fivefold, 10 fold, 50 fold, 100 fold, 500 fold, or 1000 fold lower affinity.

A subject CD47-binding domain can reduce the binding of CD47-binding-partners to CD47, including e.g., thrombospondin-1 (TSP-1), signal-regulatory protein alpha (SIRPα) and integrins (e.g., integrin avb3). For example, a subject CD47-binding domain can reduce CD47-binding-partner binding by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the degree of binding in the absence of the CD47-binding domain.

In some embodiments, a subject antibody comprises: a heavy chain FR1 region; a HCDR1; a heavy chain FR2 region; a HCDR2; a heavy chain FR3 region; a HCDR3; and a heavy chain FR4 region. In some of these embodiments, each of the FR regions is mammalian FR region, including e.g., a human FR region. In some embodiments, a subject antibody comprises: a light chain FR1 region; a LCDR1; a light chain FR2 region; a LCDR2; a light chain FR3 region; a LCDR3; and a heavy chain FR4 region. In some of these embodiments, each of the FR regions is mammalian FR region, including e.g., a human FR region.

In some embodiments, a subject antibody comprises an anti-ABCG2 heavy chain sequence that includes a charge-to-charge swap (KK) modification to the Fc region, and an anti-CD47, anti-ErbB2, anti-EGFR, anti-PD-L1 heavy chain sequence that includes a charge-to-charge swap (DD) modification, especially an anti-CD47 heavy chain sequence with a charge-to-charge swap (DD) modification, having the sequence:

QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSSASTKGPSVFPLAPSSKSTSGGTAALGCLVKDYF
PEPVTVSWNSGALTSGVHTFPAVLQSSGLYSLSSWTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRKELTKNQVSLTCLVKGFYPSDIAVEWESNGQPENNYKTTPPVLKSD
GSFFLYSKLTVDKSRWQQGNVFSCSVMHEALHNHYTQKSLSLSPG (SEQ
ID NO:50).

In some instances, the subject antibody may include an alternative heterodimeric Fc pairing strategy other than charged pair substitution.

The charge to charge swap modification refers to substitutions where one heavy chain is modified to contain K392D and K409D substitutions and the other heavy chain is modified to contained E356K and D399K substitutions. Charge pair substituted chains favor formation of heterodimer with one another. The numbering of the amino acid substitutions is per EU numbering system for Ig HCs.

Regions and/or chains of the subject antibodies may or may not be joined by one or more linker regions. Where present, the linker region can be from about 5 amino acids to about 50 amino acids in length, e.g., from about 5 aa to about 10 aa, from about 10 aa to about 15 aa, from about 15 aa to about 20 aa, from about 20 aa to about 25 aa, from about 25 aa to about 30 aa, from about 30 aa to about 35 aa, from about 35 aa to about 40 aa, from about 40 aa to about 45 aa, or from about 45 aa to about 50 aa in length.

Linkers suitable for use in a subject antibody include “flexible linkers”. If present, the linker molecules are generally of sufficient length to permit some flexible movement between linked regions. The linker molecules are generally about 6-50 atoms long. The linker molecules may also be, for example, aryl acetylene, ethylene glycol oligomers containing 2-10 monomer units, diamines, diacids, amino acids, or combinations thereof. Other linker molecules which can bind to polypeptides may be used in light of this disclosure.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)ₙ, glycine-serine polymers (including, for example, (GS)ₙ, GSGGSₙ (SEQ ID NO:51)) and GGGSₙ (SEQ ID NO:52), where n is an integer of at least one), glycine-alanine polymers, alanine-serine polymers, and other flexible linkers known in the art. Glycine and glycine-serine polymers are of interest since both of these amino acids are relatively unstructured, and therefore may serve as a neutral tether between components. Glycine polymers are of particular interest since glycine accesses significantly more phi-psi space than even alanine, and is much less restricted than residues with longer side chains (see Scheraga, Rev. Computational Chem. 11173-142 (1992)). Exemplary flexible linkers include, but are not limited to GGSG (SEQ ID NO:53), GGSGG (SEQ ID NO:54), GSGSG (SEQ ID NO:55), GSGGG (SEQ ID NO:56), GGGSG (SEQ ID NO:57), GSSSG (SEQ ID NO:58), and the like. The ordinarily skilled artisan will recognize that design of a peptide conjugated to any elements described above can include linkers that are all or partially flexible, such that the linker can include a flexible linker as well as one or more portions that confer less flexible structure.

In some embodiments, a subject antibody is “humanized.” The term “humanized antibody” refers to an antibody comprising at least one chain comprising variable region framework residues substantially from a human antibody chain (referred to as the acceptor immunoglobulin or antibody) and at least one CDR substantially from a non-human antibody (such as e.g., a rodent (e.g., mouse antibody), non-human primate, etc.), (referred to as the donor immunoglobulin or antibody). See, Queen et al., Proc. Natl. Acad. Sci. USA 86:10029 10033 (1989), U.S. Pat. No. 5,530,101, U.S. Pat. No. 5,585,089, U.S. Pat. No. 5,693,761, WO 90/07861, and U.S. Pat. No. 5,225,539. The constant region(s), if present, can also be substantially or entirely from a human immunoglobulin. In some embodiments, a subject antibody comprises one or more ABCG2 CDRs and one or more CD47 CDRs and one or more FR regions from a human antibody. Methods of making humanized antibodies are known in the art. See, e.g., U.S. Pat. No. 7,256,273.

The substitution of mouse CDRs into a human variable domain framework can result in retention of their correct spatial orientation where, e.g., the human variable domain framework adopts the same or similar conformation to the mouse variable framework from which the CDRs originated. This can be achieved by obtaining the human variable domains from human antibodies whose framework sequences exhibit a high degree of sequence identity with the murine variable framework domains from which the CDRs were derived. The heavy and light chain variable framework regions can be derived from the same or different human antibody sequences. The human antibody sequences can be the sequences of naturally occurring human antibodies or can be consensus sequences of several human antibodies. See Kettleborough et al., Protein Engineering 4:773 (1991); Kolbinger et al., Protein Engineering 6:971 (1993).

Having identified the complementarity determining regions of the murine donor immunoglobulin and appropriate human acceptor immunoglobulins, the next step is to determine which, if any, residues from these components should be substituted to optimize the properties of the resulting humanized antibody. In general, substitution of human amino acid residues with murine should be minimized, because introduction of murine residues increases the risk of the antibody eliciting a human-anti-mouse-antibody (HAMA) response in humans. Art-recognized methods of determining immune response can be performed to monitor a HAMA response in a particular patient or during clinical trials. Patients administered humanized antibodies can be given an immunogenicity assessment at the beginning and throughout the administration of said therapy. The HAMA response is measured, for example, by detecting antibodies to the humanized therapeutic reagent, in serum samples from the patient using a method known to one in the art, including surface plasmon resonance technology (BIACORE) and/or solid-phase ELISA analysis. In many embodiments, a subject humanized antibody does not substantially elicit a HAMA response in a human subject.

Certain amino acids from the human variable region framework residues are selected for substitution based on their possible influence on CDR conformation and/or binding to antigen. The unnatural juxtaposition of murine CDR regions with human variable framework region can result in unnatural conformational restraints, which, unless corrected by substitution of certain amino acid residues, lead to loss of binding affinity.

The selection of amino acid residues for substitution can be determined, in part, by computer modeling. Computer hardware and software for producing three-dimensional images of immunoglobulin molecules are known in the art. In general, molecular models are produced starting from solved structures for immunoglobulin chains or domains thereof. The chains to be modeled are compared for amino acid sequence similarity with chains or domains of solved three-dimensional structures, and the chains or domains showing the greatest sequence similarity is/are selected as starting points for construction of the molecular model. Chains or domains sharing at least 50% sequence identity are selected for modeling, and preferably those sharing at least 60%, 70%, 80%, 90% sequence identity or more are selected for modeling. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

CDR and framework regions are as defined by Kabat, Sequences of Proteins of Immunological Interest (National Institutes of Health, Bethesda, Md., 1987 and 1991). An alternative structural definition has been proposed by Chothia et al., J. Mol. Biol. 196:901 (1987); Nature 342:878 (1989); and J. Mol. Biol. 186:651 (1989) (collectively referred to as “Chothia”). When framework residues, as defined by Kabat, supra, constitute structural loop residues as defined by Chothia, supra, the amino acids present in the mouse antibody may be selected for substitution into the humanized antibody. Residues which are “adjacent to a CDR region” include amino acid residues in positions immediately adjacent to one or more of the CDRs in the primary sequence of the humanized immunoglobulin chain, for example, in positions immediately adjacent to a CDR as defined by Kabat, or a CDR as defined by Chothia (See e.g., Chothia and Lesk JMB 196:901 (1987)). These amino acids are particularly likely to interact with the amino acids in the CDRs and, if chosen from the acceptor, to distort the donor CDRs and reduce affinity. Moreover, the adjacent amino acids may interact directly with the antigen (Amit et al., Science, 233:747 (1986)) and selecting these amino acids from the donor may be desirable to keep all the antigen contacts that provide affinity in the original antibody.

In some embodiments, a subject antibody comprises scFv multimers. For example, in some embodiments, a subject antibody is an scFv dimer (e.g., comprises two tandem scFv (scFv₂)), an scFv trimer (e.g., comprises three tandem scFv (scFv₃)), an scFv tetramer (e.g., comprises four tandem scFv (scFv₄)), or is a multimer of more than four scFv (e.g., in tandem). The scFv monomers can be linked in tandem via linkers of from about 2 amino acids to about 10 amino acids in length, e.g., 2 aa, 3 aa, 4 aa, 5 aa, 6 aa, 7 aa, 8 aa, 9 aa, or 10 aa in length. Suitable linkers include, e.g., (Gly)ₓ (SEQ ID NO:69), where x is an integer from 2 to 10. Other suitable linkers are those discussed above. In some embodiments, each of the scFv monomers in a subject scFV multimer is humanized, as described above.

In some embodiments, a subject antibody comprises a constant region of an immunoglobulin (e.g., an Fc region). The Fc region, if present, can be a human Fc region. If constant regions are present, the antibody can contain both light chain and heavy chain constant regions. Suitable heavy chain constant region include CH1, hinge, CH2, CH3, and CH4 regions. The antibodies described herein include antibodies having all types of constant regions, including IgM, IgG, IgD, IgA and IgE, and any isotype, including IgG1, IgG2, IgG3 and IgG4. An example of a suitable heavy chain Fc region is a human isotype IgG1 Fc. Light chain constant regions can be lambda or kappa. A subject antibody (e.g., a subject humanized antibody) can comprise sequences from more than one class or isotype. Antibodies can be expressed as tetramers containing two light and two heavy chains, as separate heavy chains, light chains, as Fab, Fab′ F(ab′)2, and Fv, or as single chain antibodies in which heavy and light chain variable domains are linked through a spacer.

In some embodiments, a subject antibody comprises a free thiol (-SH) group at the carboxyl terminus, where the free thiol group can be used to attach the antibody to a second polypeptide (e.g., another antibody, including a subject antibody), a scaffold, a carrier, etc.

A subject antibody can be covalently linked to a second moiety (e.g., a lipid, a polypeptide other than a subject antibody, a synthetic polymer, a carbohydrate, and the like) using for example, glutaraldehyde, a homobifunctional cross-linker, or a heterobifunctional cross-linker. Glutaraldehyde cross-links polypeptides via their amino moieties. Homobifunctional cross-linkers (e.g., a homobifunctional imidoester, a homobifunctional N-hydroxysuccinimidyl (NHS) ester, or a homobifunctional sulfhydryl reactive cross-linker) contain two or more identical reactive moieties and can be used in a one-step reaction procedure in which the cross-linker is added to a solution containing a mixture of the polypeptides to be linked. Homobifunctional NHS ester and imido esters cross-link amine containing polypeptides. In a mild alkaline pH, imido esters react only with primary amines to form imidoamides, and overall charge of the cross-linked polypeptides is not affected. Homobifunctional sulfhydryl reactive cross-linkers includes bismaleimidhexane (BMH), 1,5-difluoro-2,4-dinitrobenzene (DFDNB), and 1,4-di-(3′,2′-pyridyldithio) propinoamido butane (DPDPB).

### Compositions and Formulations

The present disclosure provides a composition comprising a subject antibody. A subject antibody composition can comprise, in addition to a subject antibody, one or more of: a salt, e.g., NaCl, MgCl₂, KCI, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like.

Compositions of the present disclosure also include pharmaceutical compositions that include a multi-specific antibody described herein. In general, a formulation comprises an effective amount of the subject antibody. An “effective amount” means a dosage sufficient to produce a desired result, e.g., reduction in a cancer of a subject, reduction in the growth rate of a cancer in a subject, amelioration of a symptom of cancer, and the like. Generally, the desired result is at least a reduction in a symptom of a cancer, reduction in the growth of a cancer, reduction in the size of a cancer, etc., as compared to a control. A subject antibody can be delivered, or be formulated, in such a manner as to avoid the blood-brain barrier. In some instances, an antibody may include a delivery enhancer, including where such enhancers may facilitate crossing of the blood-brain barrier, increased permeability, e.g., allowing for efficient transdermal delivery, and the like. Useful delivery enhancers include but are not limited to e.g., cereport, regadenoson, borneol, puerarin, propylene glycols, oleic acid, azone, N-methyl pyrrolidone, Tween 80, limonene, lipid-based nanoparticles (NPs), liposomes, niosomes, transfersomes, ethosomes, dendrimers, micellar NPs, polymeric nanostructures, metallic nanostructures, magnetic nanostructures, recombinant human hyaluronidase, and the like.

In the subject methods, a subject antibody can be administered to the host using any convenient means capable of resulting in the desired therapeutic effect or diagnostic effect. Thus, the agent can be incorporated into a variety of formulations for therapeutic administration. More particularly, a subject antibody can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, a subject antibody can be administered in conjunction with a pharmaceutically acceptable excipient, or they may also be used alone or in appropriate association, as well as in combination, with other pharmaceutically active compounds. The following methods and excipients are merely exemplary and are in no way limiting.

For oral preparations, a subject antibody can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. In some instances, oral delivery of antibodies may be enhanced through complexation of the antibody with an appropriate hydrogel.

A subject antibody can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions comprising a subject antibody are prepared by mixing the antibody having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration; see also Chen (1992) Drug Dev Ind Pharm 18, 1311-54.

Exemplary antibody concentrations in a subject pharmaceutical composition may range from about 1 mg/mL to about 200 mg/ml or from about 50 mg/mL to about 200 mg/mL, or from about 150 mg/mL to about 200 mg/mL.

An aqueous formulation of the antibody may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the antibody formulation to modulate the tonicity of the formulation. Exemplary tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 nM.

A surfactant may also be added to the antibody formulation to reduce aggregation of the formulated antibody and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20®) and polysorbate 80 (sold under the trademark Tween 80®). Examples of suitable polyethylene-polypropylene copolymers are those sold under the names Pluronic® F68 or Poloxamer 188®. Examples of suitable Polyoxyethylene alkyl ethers are those sold under the trademark Brij®. Exemplary concentrations of surfactant may range from about 0.001 % to about 1% w/v.

A lyoprotectant may also be added in order to protect the labile active ingredient (e.g. a protein) against destabilizing conditions during the lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, a subject formulation includes a subject antibody, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

For example, a subject formulation can be a liquid or lyophilized formulation suitable for parenteral administration, and can comprise: about 1 mg/mL to about 200 mg/mL of a subject antibody; about 0.001 % to about 1 % of at least one surfactant; about 1 mM to about 100 mM of a buffer; optionally about 10 mM to about 500 mM of a stabilizer; and about 5 mM to about 305 mM of a tonicity agent; and has a pH of about 4.0 to about 7.0.

As another example, a subject parenteral formulation is a liquid or lyophilized formulation comprising: about 1 mg/mL to about 200 mg/mL of a subject antibody; 0.04% Tween 20 w/v; 20 mM L-histidine; and 250 mM Sucrose; and has a pH of 5.5.

As another example, a subject parenteral formulation comprises a lyophilized formulation comprising: 1) 15 mg/mL of a subject antibody; 0.04% Tween 20 w/v; 20 mM L-histidine; and 250 mM sucrose; and has a pH of 5.5; or 2) 75 mg/mL of a subject antibody; 0.04% Tween 20 w/v; 20 mM L-histidine; and 250 mM sucrose; and has a pH of 5.5;or 3) 75 mg/mL of a subject antibody; 0.02% Tween 20 w/v; 20 mM L-histidine; and 250 mM Sucrose; and has a pH of 5.5; or 4) 75 mg/mL of a subject antibody; 0.04% Tween 20 w/v; 20 mM L-histidine; and 250 mM trehalose; and has a pH of 5.5; or 6) 75 mg/mL of a subject antibody; 0.02% Tween 20 w/v; 20 mM L-histidine; and 250 mM trehalose; and has a pH of 5.5.

As another example, a subject parenteral formulation is a liquid formulation comprising:1) 7.5 mg/mL of a subject antibody; 0.022% Tween 20 w/v; 120 mM L-histidine; and 250 125 mM sucrose; and has a pH of 5.5; or 2) 37.5 mg/mL of a subject antibody; 0.02% Tween 20 w/v; 10 mM L-histidine; and 125 mM sucrose; and has a pH of 5.5; or 3) 37.5 mg/mL of a subject antibody; 0.01 % Tween 20 w/v; 10 mM L-histidine; and 125 mM sucrose; and has a pH of 5.5; or 4) 37.5 mg/mL of a subject antibody; 0.02% Tween 20 w/v; 10 mM L-histidine; 125 mM trehalose; and has a pH of 5.5; or 5) 37.5 mg/mL of a subject antibody; 0.01 % Tween 20 w/v; 10 mM L-histidine; and 125 mM trehalose; and has a pH of 5.5; or 6) 5 mg/mL of a subject antibody; 0.02% Tween 20 w/v; 20 mM L-histidine; and 250 mM trehalose; and has a pH of 5.5; or 7) 75 mg/mL of a subject antibody; 0.02% Tween 20 w/v; 20 mM L-histidine; and 250 mM mannitol; and has a pH of 5.5; or 8) 75 mg/mL of a subject antibody; 0.02% Tween 20 w/v; 20 mM L histidine; and 140 mM sodium chloride; and has a pH of 5.5;or 9) 150 mg/mL of a subject antibody; 0.02% Tween 20 w/v; 20 mM L-histidine; and 250 mM trehalose; and has a pH of 5.5; or 10) 150 mg/mL of a subject antibody; 0.02% Tween 20 w/v; 20 mM L-histidine; and 250 mM mannitol; and has a pH of 5.5; or 11) 150 mg/mL of a subject antibody; 0.02% Tween 20 w/v; 20 mM L-histidine; and 140 mM sodium chloride; and has a pH of 5.5; or 12) 10 mg/mL of a subject antibody; 0.01 % Tween 20 w/v; 20 mM L-histidine; and 40 mM sodium chloride; and has a pH of 5.5.

A subject antibody can be utilized in aerosol formulation to be administered via inhalation. A subject antibody can be formulated into pressurized acceptable propellants such as dichlorodifluoromethane, propane, nitrogen and the like.

Furthermore, a subject antibody can be made into suppositories by mixing with a variety of bases such as emulsifying bases or water-soluble bases. A subject antibody can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise a subject antibody in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for a subject antibody may depend on the particular antibody employed and the effect to be achieved, and the pharmacodynamics associated with each antibody in the host.

Other modes of administration will also find use with the subject invention. For instance, a subject antibody can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), e.g., about 1% to about 2%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

A subject antibody can be administered as an injectable formulation. Typically, injectable compositions are prepared as liquid solutions or suspensions; solid forms suitable for solution in, or suspension in, liquid vehicles prior to injection may also be prepared. The preparation may also be emulsified or the antibody encapsulated in liposome vehicles.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington’s Pharmaceutical Sciences, Mack Publishing Company, Easton, Pennsylvania, 17th edition, 1985. The composition or formulation to be administered will, in any event, contain a quantity of a subject antibody adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, a subject antibody is formulated in a controlled release formulation. Sustained-release preparations may be prepared using methods well known in the art. Suitable examples of sustained-release preparations include semipermeable matrices of solid hydrophobic polymers containing the antibody in which the matrices are in the form of shaped articles, e.g. films or microcapsules. Examples of sustained-release matrices include polyesters, copolymers of L-glutamic acid and ethyl-L-glutamate, non-degradable ethylenevinyl acetate, hydrogels, polylactides, degradable lactic acid-glycolic acid copolymers and poly-D-(-)-3-hydroxybutyric acid. Possible loss of biological activity and possible changes in immunogenicity of antibodies comprised in sustained-release preparations may be prevented by using appropriate additives, by controlling moisture content and by developing specific polymer matrix compositions.

Controlled release within the scope of this invention can be taken to mean any one of a number of extended release dosage forms. The following terms may be considered to be substantially equivalent to controlled release, for the purposes of the present invention: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.).

### Dosages

A suitable dosage can be determined by an attending physician or other qualified medical personnel, based on various clinical factors. As is well known in the medical arts, dosages for any one patient depend upon many factors, including the patient’s size, body surface area, age, the particular compound to be administered, sex of the patient, time, and route of administration, general health, and other drugs being administered concurrently. A subject antibody may be administered in amounts between 1 ng/kg body weight and 20 mg/kg body weight per dose, e.g. between 0.1 mg/kg body weight to 10 mg/kg body weight, e.g. between 0.5 mg/kg body weight to 5 mg/kg body weight; however, doses below or above this exemplary range are envisioned, especially considering the aforementioned factors. If the regimen is a continuous infusion, it can also be in the range of 1 µg to 10 mg per kilogram of body weight per minute.

Those of skill will readily appreciate that dose levels can vary as a function of the specific antibody, the severity of the symptoms and the susceptibility of the subject to side effects. Preferred dosages for a given compound are readily determinable by those of skill in the art by a variety of means.

### Routes of Administration

A subject antibody is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, subcutaneous, intradermal, topical application, intravenous, intraarterial, rectal, nasal, oral, and other enteral and parenteral routes of administration. Routes of administration may be combined, if desired, or adjusted depending upon the antibody and/or the desired effect. A subject antibody composition can be administered in a single dose or in multiple doses. In some embodiments, a subject antibody composition is administered orally. In some embodiments, a subject antibody composition is administered via an inhalational route. In some embodiments, a subject antibody composition is administered intranasally. In some embodiments, a subject antibody composition is administered locally. In some embodiments, a subject antibody composition is administered intracranially. In some embodiments, a subject antibody composition is administered intravenously.

The agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of a subject antibody. Where systemic delivery is desired, administration typically involves invasive or systemically absorbed topical or mucosal administration of pharmaceutical preparations.

A subject antibody can also be delivered to the subject by enteral administration. Enteral routes of administration include, but are not necessarily limited to, oral and rectal (e.g., using a suppository) delivery.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as cancer and/or the growth of a cancer and pain associated therewith. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts (wherein the term “host” is used interchangeably herein with the terms “subject,” “individual,” and “patient”) are treatable according to the subject methods. Generally, such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the hosts will be humans.

Kits with unit doses of a subject antibody, e.g. in oral or injectable doses, are provided. In some embodiments, in addition to the containers containing the unit doses will be an informational package insert describing the use and attendant benefits of the antibody in treating pathological condition of interest.

### Nucleic Acids

The present disclosure provides nucleic acids comprising nucleotide sequences encoding a subject antibody. A nucleotide sequence encoding a subject antibody can be operably linked to one or more regulatory elements, such as a promoter and enhancer, that allow expression of the nucleotide sequence in the intended target cells (e.g., a cell that is genetically modified to synthesize and/or secrete the encoded antibody).

Suitable promoter and enhancer elements are known in the art. For expression in a bacterial cell, suitable promoters include, but are not limited to, lacl, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

A nucleotide sequence encoding a subject antibody can be present in an expression vector and/or a cloning vector. Where a subject antibody comprises two or more separate polypeptides, nucleotide sequences encoding the two polypeptides can be cloned in the same or separate vectors. Separate polypeptides may be expressed from a single nucleic acid or single vector using various strategies, such as separate promoters, one or more internal ribosomal entry sites (IRES), one or more self-cleaving sequences (e.g., 2A cleavage sequences, e.g., P2A, T2A, E2A, and F2A), combinations thereof, and the like. An expression vector can include a selectable marker, an origin of replication, and other features that provide for replication and/or maintenance of the vector.

Large numbers of suitable vectors and promoters are known to those of skill in the art; many are commercially available for generating a subject recombinant construct. The following vectors are provided by way of example. Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

As noted above, a subject nucleic acid comprises a nucleotide sequence encoding a subject multi-specific antibody. A subject nucleic acid can comprise a nucleotide sequence encoding heavy- and light-chain CDRs. In some embodiments, a subject nucleic acid comprises a nucleotide sequence encoding heavy- and/or light-chain CDRs, where the CDR-encoding sequences are interspersed with FR-encoding nucleotide sequences. In some embodiments, a subject nucleic acid comprises a nucleotide sequence encoding heavy-and/or light-chain CD47 CDRs, where the CDR-encoding sequences are interspersed with FR-encoding nucleotide sequences. In some embodiments, the FR-encoding nucleotide sequences are human FR-encoding nucleotide sequences.

In some embodiments, a subject nucleic acid comprises a nucleotide sequence encoding an amino acid sequence, wherein the amino acid sequence has at least about 75%, at least about 80%, at least about 85%, at least about 90%, at least about 95%, at least about 98%, at least about 99%, or 100% sequence identity, to the amino acid sequences provided herein.

Nucleic acids, e.g., as described herein, may, in some instances, be introduced into a cell, e.g., by contacting the cell with the nucleic acid. Cells with introduced nucleic acids will generally be referred to herein as genetically modified cells. Various methods of nucleic acid delivery may be employed including but not limited to e.g., naked nucleic acid delivery, viral delivery, chemical transfection, biolistics, and the like.

### Cells

The present disclosure provides isolated genetically modified cells (e.g., in vitro cells, ex vivo cells, cultured cells, etc.) that are genetically modified with a subject nucleic acid. In some embodiments, a subject isolated genetically modified cell can produce a subject antibody. In some instances, a genetically modified cell can deliver an antibody, e.g., to a subject in need thereof. In some instances, a genetically modified cell may be used in the production, screening, and/or discovery of multi-specific antibodies. Genetically modified cells may also, in some instances, include cells where endogenous gene expression has been reduced, e.g., inhibited, knocked-down, etc., or abolished, e.g., knocked-out. Genetically modified cells may also, in some instances, include cells where expression of a gene has been enhanced, e.g., the expression of an endogenous gene is increased or the expression of a heterologous gene is increased.

Suitable cells include eukaryotic cells, such as a mammalian cell, an insect cell, a yeast cell; and prokaryotic cells, such as a bacterial cell. Introduction of a subject nucleic acid into the host cell can be effected, for example by calcium phosphate precipitation, DEAE dextran mediated transfection, liposome-mediated transfection, electroporation, or other known method.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In some instances, useful mammalian cells may include cells derived from a mammalian tissue or organ. In some instances, cells employed are kidney cells, including e.g., kidney cells of an established kidney cell line, such as HEK 293T cells.

Suitable yeast cells or fungi or algae cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like.

Suitable prokaryotic cells include, but are not limited to, any of a variety of laboratory strains of Escherichia coli, Lactobacillus sp., Salmonella sp., Shigella sp., and the like. See, e.g., Carrier et al. (1992) J. Immunol. 148:1176-1181; U.S. Pat. No. 6,447,784; and Sizemore et al. (1995) Science 270:299-302. Examples of Salmonella strains which can be employed in the present invention include, but are not limited to, Salmonella typhi and S. typhimurium. Suitable Shigella strains include, but are not limited to, Shigella flexneri, Shigella sonnei, and Shigella disenteriae. Typically, the laboratory strain is one that is non-pathogenic. Non-limiting examples of other suitable bacteria include, but are not limited to, Bacillus subtilis, Pseudomonas pudita, Pseudomonas aeruginosa, Pseudomonas mevalonii, Rhodobacter sphaeroides, Rhodobacter capsulatus, Rhodospirillum rubrum, Rhodococcus sp., and the like. In some embodiments, the host cell is Escherichia coli.

In some instances, cells of the present disclosure may be immune cells. As used herein, the term “immune cells” generally includes white blood cells (leukocytes) which are derived from hematopoietic stem cells (HSC) produced in the bone marrow. “Immune cells” includes, e.g., lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), T-regulatory cells (Treg) and gamma-delta T cells. A “cytotoxic cell” includes CD8+ T cells, natural-killer (NK) cells, and neutrophils, which cells are capable of mediating cytotoxicity responses.

In some instances, useful cells expressing a multi-specific antibody of the present disclosure may include producer T cells. Nonlimiting examples of producer T cells include those described in Tsai & Davila Oncoimmunology. (2016) 5(5): e1122158; the disclosure of which is incorporated herein by reference in its entirety. Producer T cells engineered to include nucleic acid sequence encoding a multi-specific antibody of the present disclosure may, in some instances, be employed to deliver the antibody to a subject in need thereof.

Cells of the present disclosure also include cells genetically modified to change and/or amend expression of one or more of ABCG2 and TAA (e.g., CD47) in the cell. Such modified cells are useful for various purposes including assaying the binding of multi-specific antibodies, including but not limited to those produced according to the description and methods provided herein. In some instances, ABCG2 may be knocked out or knocked down in a subject cell line. In some instances, TAA (e.g., CD47) may be knocked out or knocked down in a subject cell line. In some instances, ABCG2 may be constitutively or inducibly overexpressed in a subject cell line. In some instances, TAA (e.g., CD47) may be constitutively or inducibly overexpressed in a subject cell line. In some instances, both ABCG2 and TAA (e.g., CD47) may knocked down, knocked out, or constitutively or inducibly overexpressed in a subject cell line. Any convenient and appropriate method for knockdown, knockout and/or overexpression may be employed. Introduced nucleic acid may be stably integrated or present transiently.

In some embodiments, cells of the present disclosure include a genetically modified human cell line that expresses TAA (e.g., CD47) and includes an exogenous nucleic acid comprising a sequence encoding ABCG2 for overexpression of ABCG2. In such cells TAA (e.g., CD47) expression may by endogenous or exogenously derived (i.e., introduced) and ABCG2 expression may be stable or transient. In some instances, cells lines of the present disclosure, that express TAA (e.g., CD47), may be configured to produce a genetically modified human cell expressing TAA (e.g., CD47) and stably overexpressing ABCG2.

Cells and cell lines of the present disclosure may be cultured, including e.g., through use of culture methods described herein. In some instances, a cell, into which nucleic acid have been introduced to genetically modify the cell, may be cultured to produce a cell line. Useful cells lines may include but are not limited to e.g., genetically modified cell lines, including human cell lines, expressing TAA (e.g., CD47) and stably over expressing ABCG2.

Cells of the present disclosure, and cell lines thereof, may be employed in various methods of the disclosure, e.g., as test samples, controls, and the like. For example, in some instances cells in which ABCG2 and/or TAA (e.g., CD47) have been knocked out and/or knocked down may be employed as reference cells, e.g., to which the binding of a multi-specific antibody of the present disclosure may be compared. Other useful reference cells include but are not limited to e.g., non-cancerous cells, as well as normal cells and cells expressing normal levels of various proteins, including normal level of ABCG2 and/or TAA (e.g., CD47).

### Methods

As summarized above, methods of the present disclosure include methods of contacting a cell with an antibody of the present disclosure, methods of treating a subject according to a method that involves administering to the subject an antibody of the present disclosure, methods of making elements described in the instant application, including e.g., multi-specific antibodies, compositions and formulations, nucleic acids, expression vectors, cells, and the like.

As summarized above, methods of the present disclosure include contacting a cancer cell with the multi-specific antibody of the present disclosure, e.g., to facilitate and/or enhance killing of the cancer cell. In some instances, killing of the cancer cell is mediated by an immune response or immune cell acting upon the cancer cell as a result of opsonization of the cancer cell by bispecific targeting when the two targets are co-expressed on the cancer cell. In some instances, killing of the cancer cell is mediated by an immune response or immune cell acting upon the cancer cell, e.g., as a result of masking or antagonizing of a TAA (e.g., CD47) epitope present on the surface of the cancer cell by the multi-specific antibody. In some instances, killing of the cancer cell is mediated by inhibition of cellular efflux of the cancer cell, e.g., as a result of ABCG2 antagonism on the cancer cell by the multi-specific antibody. In some instances, the cell contacted with the multi-specific antibody may be a multidrug resistant cancer cell. Methods that involve contacting a cancer cell with a multi-specific antibody of the present disclosure may or may not include contacting the cancer cell with an additional therapy or active agent, including e.g., a chemotherapeutic, an immunotherapy, radiation therapy, or the like.

Contacting a cancer cell with a multi-specific antibody of the present disclosure will generally enhance the killing of the cancer cell, e.g., as compared to the level of killing of the cancer cell in the absence of the multi-specific antibody. In some instances, where an additional active agent is employed, enhanced killing of the cancer cell may be seen as compared to the level of killing observed using the additional active agent alone. The amount of enhancement of cancer cell killing attributable to the multi-specific antibody will vary and may range from at least a 5% increase in cancer cell killing to at least 90% or more, including but not limited to e.g., at least 10%, at least 20%, at least 30%, at least 40%, at least 50%, at least 60%, at least 70%, at least 80%, etc. Such increases may be compared to contacting with one or more additional active agents alone.

Enhanced killing of a cancer cell may be assessed by a variety of means including but not limited to e.g., observational studies, in vitro cell-based cytotoxicity assays, flow cytometry, cell viability labeling (e.g., using one or more cell viability stains), and the like.

### Treatment Methods

The present disclosure provides methods of treating a cancer, the methods generally involving administering to an individual in need thereof (e.g., an individual having a cancer) an effective amount of a subject multi-specific antibody, alone (e.g., in monotherapy) or in combination (e.g., in combination therapy) with one or more additional therapeutic agents. Administration of a multi-specific antibody of the present disclosure may be performed by any convenient and appropriate route of delivery.

Aspects of the present disclosure include the bispecific antibody molecule according to the preceding section of the specification for use in a method of treating cancer in a subject, the method comprising administering the antibody to the subject. The method comprises administering the antibody in combination with at least one additional active agent, wherein the at least one additional active agent comprises a chemotherapeutic agent, an inhibitor of a multidrug resistance transporter, an immunotherapy agent, or a combination thereof. In certain aspects, the at least one additional active agent is a chemotherapeutic agent, optionally wherein the chemotherapeutic agent is a taxol, a vinca alkaloid, an anthracycline, taxol, a vinca alkaloid, an anthracycline, Etoposide, Mitoxantrone, or Methotrexate. Substrates for ABCG2 include topoisomerase II inhibitors (e.g., Mitoxantrone, anthracyclines: Doxorubicin, Epirubicin, etc.); topoisomerase I inhibitors (e.g., Camptothecin analogs: Topotecan, Gimatecan, etc.); Tyrosine kinase inhibitors (e.g., Gefitinib) and the like.

Also disclosed herein is a chemotherapy agent for use in a method of treating cancer in a subject, the method comprising administering the chemotherapy agent in combination with the antibody described herein to the subject, optionally wherein the chemotherapy agent is a taxol, a vinca alkaloid, an anthracycline, Etoposide, Mitoxantrone, or Methotrexate.

Accordingly, administration includes but is not limited to e.g., delivery of the antibody by injection, delivery of the antibody by infusion, delivery of a nucleic acid or expression vector encoding the multi-specific antibody, delivery of the antibody by administering to the subject a cell that expresses and secretes the multi-specific antibody, and the like. Administration of an agent, a nucleic acid encoding an agent, a cell expressing an agent, etc. may include contacting with the agent, contacting with the nucleic acid, contacting with the cell, etc.

In some embodiments, an effective amount of a subject multi-specific antibody is an amount that, when administered alone (e.g., in monotherapy) or in combination (e.g., in combination therapy) with one or more additional therapeutic agents, in one or more doses, is effective to reduce an adverse symptom of cancer by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the severity of the adverse symptom in the absence of treatment with the antibody.

In some embodiments, an effective amount of a subject multi-specific antibody is an amount that, when administered alone (e.g., in monotherapy) or in combination (e.g., in combination therapy) with one or more additional therapeutic agents, in one or more doses, is effective to improve the cancer (i.e., slow the growth of the cancer, stop the growth of the cancer, reverse the growth of the cancer, kill cancer cells (including tumor cells, or the like) in the individual being treated. For example, an effective amount of a subject antibody can reduce a cancer growth rate or reduce a cancer size in an individual by at least about 5%, at least about 10%, at least about 15%, at least about 20%, at least about 25%, at least about 30%, at least about 40%, at least about 50%, or more, compared to in the absence of treatment with the multi-specific antibody.

In some instances, a subject may be treated systemically, including with the subject multi-specific antibody, with or without one or more additional reagents. By “systemic treatment”, as used herein, is meant a treatment that is not directed solely to target a specific tumor (such as e.g., a primary tumor or a defined secondary tumor) or a specific cancer containing tissue (such as e.g., the liver in the case of liver cancer, the blood in the case of a blood cancer, etc.). Systemic treatments will generally be directed to the subject’s body as a whole and may include but are not limited to e.g., systemic radiation therapy, systemic chemotherapy, systemic immunotherapy, combinations thereof and the like.

In some instances, a subject may be treated locally, including with the subject multi-specific antibody, with or without one or more additional reagents. By “local treatment”, as used herein, is meant a treatment that is specifically directed to the location of a tumor (such as e.g., a primary tumor or a defined secondary tumor) or specifically directed to a cancer containing tissue (such as e.g., the liver in the case of liver cancer, the blood in the case of a blood cancer, etc.). In some instances, local treatment may also be administered in such a way as to affect the environment surrounding a tumor, such as tissue surrounding the tumor, such as tissue immediately adjacent to the tumor. Local treatment will generally not affect or not be targeted to tissues distant from the site of cancer including the site of a tumor, such as a primary tumor. Useful local treatments that may be administered in addition to or in combination with a subject multi-specific antibody, e.g., include but are not limited to surgery, local radiation therapy, local cryotherapy, local laser therapy, local topical therapy, combinations thereof, and the like.

In some embodiments, a subject treatment method involves administering a subject multi-specific antibody and one or more additional therapeutic agents. Suitable additional therapeutic agents include, but are not limited to, chemotherapeutic agents, radiation therapy reagents, immunotherapy reagents, other antibody or multi-specific antibody agents, and the like. Additional therapies that may be administered to a subject before, during or after a subject administering a multi-specific antibody of the present disclosure will vary depending on numerous factors including e.g., the type of cancer, the subject’s medical history, general state of health and/or any co-morbidities, and the like. Useful cancer therapies include but are not limited to e.g., radiation therapy, chemotherapy, immunotherapy, and the like.

Radiation therapy includes, but is not limited to, x-rays or gamma rays that are delivered from either an externally applied source such as a beam, or by implantation of small radioactive sources.

Suitable antibodies for use in cancer treatment include, but are not limited to, naked antibodies, e.g., trastuzumab (Herceptin) , bevacizumab (Avastin®), cetuximab (Erbitux®), panitumumab (Vectibix®), Ipilimumab (Yervoy®), rituximab (Rituxan), alemtuzumab (Lemtrada®), Ofatumumab (Arzerra®), Oregovomab (OvaRex®), Lambrolizumab (MK-3475), pertuzumab (Perjeta®), ranibizumab (Lucentis®), atezolizumab (Tecentriq®), etc., and conjugated antibodies, e.g., gemtuzumab ozogamicin (Mylortarg®), Brentuximab vedotin (Adcetris®), 90Y-labelled ibritumomab tiuxetan (Zevalin®), 131I-labelled tositumoma (Bexxar®), etc. Suitable antibodies for use in cancer treatment also include, but are not limited to, antibodies raised against tumor-associated antigens. Such antigens include, but are not limited to, CD20, CD30, CD33, CD52, EpCAM, CEA, gpA33, Mucins, TAG-72, CAIX, PSMA, Folate-binding protein, Gangliosides (e.g., GD2, GD3, GM2, etc.), Le y , VEGF, VEGFR, Integrin alpha-V-beta-3, Integrin alpha-5-beta-1, EGFR, ERBB2, ERBB3, PD-L1, MET, IGF1R, EPHA3, TRAILR1, TRAILR2, RANKL, FAP, Tenascin, etc.

Conventional cancer therapies also include targeted therapies for cancer including but not limited to e.g., Ado-trastuzumab emtansine (Kadcyla) targeting HER2 (ERBB2/neu) (approved for use in Breast cancer); Afatinib (Gilotrif) targeting EGFR (HER1/ERBB1), HER2 (ERBB2/neu) (approved for use in Non-small cell lung cancer); Aldesleukin (Proleukin) targeting (approved for use in Renal cell carcinoma, Melanoma); Alectinib (Alecensa) targeting ALK (approved for use in Non-small cell lung cancer); Alemtuzumab (Campath) targeting CD52 (approved for use in B-cell chronic lymphocytic leukemia); Atezolizumab (Tecentriq) targeting PD-L1 (approved for use in Urothelial carcinoma, Non-small cell lung cancer); Avelumab (Bavencio) targeting PD-L1 (approved for use in Merkel cell carcinoma); Axitinib (Inlyta) targeting KIT, PDGFRβ, VEGFR1/2/3 (approved for use in Renal cell carcinoma); Belimumab (Benlysta) targeting BAFF (approved for use in Lupus erythematosus); Belinostat (Beleodaq) targeting HDAC (approved for use in Peripheral T-cell lymphoma); Bevacizumab (Avastin) targeting VEGF ligand (approved for use in Cervical cancer, Colorectal cancer, Fallopian tube cancer, Glioblastoma, Non-small cell lung cancer, Ovarian cancer, Peritoneal cancer, Renal cell carcinoma); Blinatumomab (Blincyto) targeting CD19/CD3 (approved for use in Acute lymphoblastic leukemia (precursor B-cell)); Bortezomib (Velcade) targeting Proteasome (approved for use in Multiple myeloma, Mantle cell lymphoma); Bosutinib (Bosulif) targeting ABL (approved for use in Chronic myelogenous leukemia); Brentuximab vedotin (Adcetris) targeting CD30 (approved for use in Hodgkin lymphoma, Anaplastic large cell lymphoma); Brigatinib (Alunbrig) targeting ALK (approved for use in Non-small cell lung cancer (ALK+)); Cabozantinib (Cabometyx, Cometriq) targeting FLT3, KIT, MET, RET, VEGFR2 (approved for use in Medullary thyroid cancer, Renal cell carcinoma); Carfilzomib (Kyprolis) targeting Proteasome (approved for use in Multiple myeloma); Ceritinib (Zykadia) targeting ALK (approved for use in Non-small cell lung cancer); Cetuximab (Erbitux) targeting EGFR (HER1/ERBB1) (approved for use in Colorectal cancer, Squamous cell cancer of the head and neck); Cobimetinib (Cotellic) targeting MEK (approved for use in Melanoma); Crizotinib (Xalkori) targeting ALK, MET, ROS1 (approved for use in Non-small cell lung cancer); Dabrafenib (Tafinlar) targeting BRAF (approved for use in Melanoma, Non-small cell lung cancer); Daratumumab (Darzalex) targeting CD38 (approved for use in Multiple myeloma); Dasatinib (Sprycel) targeting ABL (approved for use in Chronic myelogenous leukemia, Acute lymphoblastic leukemia); Denosumab (Xgeva) targeting RANKL (approved for use in Giant cell tumor of the bone); Dinutuximab (Unituxin) targeting B4GALNT1 (GD2) (approved for use in Pediatric neuroblastoma); Durvalumab (Imfinzi) targeting PD-L1 (approved for use in Urothelial carcinoma); Elotuzumab (Empliciti) targeting SLAMF7 (CS1/CD319/CRACC) (approved for use in Multiple myeloma); Enasidenib (Idhifa) targeting IDH2 (approved for use in Acute myeloid leukemia); Erlotinib (Tarceva) targeting EGFR (HER1/ERBB1) (approved for use in Non-small cell lung cancer, Pancreatic cancer); Everolimus (Afinitor) targeting mTOR (approved for use in Pancreatic, gastrointestinal, or lung origin neuroendocrine tumor, Renal cell carcinoma, Nonresectable subependymal giant cell astrocytoma, Breast cancer); Gefitinib (Iressa) targeting EGFR (HER1/ERBB1) (approved for use in Non-small cell lung cancer); Ibritumomab tiuxetan (Zevalin) targeting CD20 (approved for use in Non-Hodgkin’s lymphoma); Ibrutinib (Imbruvica) targeting BTK (approved for use in Mantle cell lymphoma, Chronic lymphocytic leukemia, Waldenstrom’s macroglobulinemia); Idelalisib (Zydelig) targeting PI3Kδ (approved for use in Chronic lymphocytic leukemia, Follicular B-cell non-Hodgkin lymphoma, Small lymphocytic lymphoma); Imatinib (Gleevec) targeting KIT, PDGFR, ABL (approved for use in GI stromal tumor (KIT+), Dermatofibrosarcoma protuberans, Multiple hematologic malignancies); Ipilimumab (Yervoy) targeting CTLA-4 (approved for use in Melanoma); Ixazomib (Ninlaro) targeting Proteasome (approved for use in Multiple Myeloma); Lapatinib (Tykerb) targeting HER2 (ERBB2/neu), EGFR (HER1/ERBB1) (approved for use in Breast cancer (HER2+)); Lenvatinib (Lenvima) targeting VEGFR2 (approved for use in Renal cell carcinoma, Thyroid cancer); Midostaurin (Rydapt) targeting FLT3 (approved for use in acute myeloid leukemia (FLT3+)); Necitumumab (Portrazza) targeting EGFR (HER1/ERBB1) (approved for use in Squamous non-small cell lung cancer); Neratinib (Nerlynx) targeting HER2 (ERBB2/neu) (approved for use in Breast cancer); Nilotinib (Tasigna) targeting ABL (approved for use in Chronic myelogenous leukemia); Niraparib (Zejula) targeting PARP (approved for use in Ovarian cancer, Fallopian tube cancer, Peritoneal cancer); Nivolumab (Opdivo) targeting PD-1 (approved for use in Colorectal cancer, Head and neck squamous cell carcinoma, Hodgkin lymphoma, Melanoma, Non-small cell lung cancer, Renal cell carcinoma, Urothelial carcinoma); Obinutuzumab (Gazyva) targeting CD20 (approved for use in Chronic lymphocytic leukemia, Follicular lymphoma); Ofatumumab (Arzerra, HuMax-CD20) targeting CD20 (approved for use in Chronic lymphocytic leukemia); Olaparib (Lynparza) targeting PARP (approved for use in Ovarian cancer); Olaratumab (Lartruvo) targeting PDGFRα (approved for use in Soft tissue sarcoma); Osimertinib (Tagrisso) targeting EGFR (approved for use in Non-small cell lung cancer); Palbociclib (Ibrance) targeting CDK4, CDK6 (approved for use in Breast cancer); Panitumumab (Vectibix) targeting EGFR (HER1/ERBB1) (approved for use in Colorectal cancer); Panobinostat (Farydak) targeting HDAC (approved for use in Multiple myeloma); Pazopanib (Votrient) targeting VEGFR, PDGFR, KIT (approved for use in Renal cell carcinoma); Pembrolizumab (Keytruda) targeting PD-1 (approved for use in Classical Hodgkin lymphoma, Melanoma, Non-small cell lung cancer (PD-L1+), Head and neck squamous cell carcinoma, Solid tumors (MSI-H)); Pertuzumab (Perjeta) targeting HER2 (ERBB2/neu) (approved for use in Breast cancer (HER2+)); Ponatinib (Iclusig) targeting ABL, FGFR1-3, FLT3, VEGFR2 (approved for use in Chronic myelogenous leukemia, Acute lymphoblastic leukemia); Ramucirumab (Cyramza) targeting VEGFR2 (approved for use in Colorectal cancer, Gastric cancer or Gastroesophageal junction (GEJ) adenocarcinoma, Non-small cell lung cancer); Regorafenib (Stivarga) targeting KIT, PDGFRβ, RAF, RET, VEGFR1/2/3 (approved for use in Colorectal cancer, Gastrointestinal stromal tumors, Hepatocellular carcinoma); Ribociclib (Kisqali) targeting CDK4, CDK6 (approved for use in Breast cancer (HR+, HER2-)); Rituximab (Rituxan, Mabthera) targeting CD20 (approved for use in Non-Hodgkin’s lymphoma, Chronic lymphocytic leukemia, Rheumatoid arthritis, Granulomatosis with polyangiitis); Rituximab/hyaluronidase human (Rituxan Hycela) targeting CD20 (approved for use in Chronic lymphocytic leukemia, Diffuse large B-cell lymphoma, Follicular lymphoma); Romidepsin (Istodax) targeting HDAC (approved for use in Cutaneous T-cell lymphoma, Peripheral T-cell lymphoma); Rucaparib (Rubraca) targeting PARP (approved for use in Ovarian cancer); Ruxolitinib (Jakafi) targeting JAK1/2 (approved for use in Myelofibrosis); Siltuximab (Sylvant) targeting IL-6 (approved for use in Multicentric Castleman’s disease); Sipuleucel-T (Provenge) targeting (approved for use in Prostate cancer); Sonidegib (Odomzo) targeting Smoothened (approved for use in Basal cell carcinoma); Sorafenib (Nexavar) targeting VEGFR, PDGFR, KIT, RAF (approved for use in Hepatocellular carcinoma, Renal cell carcinoma, Thyroid carcinoma); Temsirolimus (Torisel) targeting mTOR (approved for use in Renal cell carcinoma); Tositumomab (Bexxar) targeting CD20 (approved for use in Non-Hodgkin’s lymphoma); Trametinib (Mekinist) targeting MEK (approved for use in Melanoma, Non-small cell lung cancer); Trastuzumab (Herceptin) targeting HER2 (ERBB2/neu) (approved for use in Breast cancer (HER2+), Gastric cancer (HER2+)); Vandetanib (Caprelsa) targeting EGFR (HER1/ERBB1), RET, VEGFR2 (approved for use in Medullary thyroid cancer); Vemurafenib (Zelboraf) targeting BRAF (approved for use in Melanoma); Venetoclax (Venclexta) targeting BCL2 (approved for use in Chronic lymphocytic leukemia); Vismodegib (Erivedge) targeting PTCH, Smoothened (approved for use in Basal cell carcinoma); Vorinostat (Zolinza) targeting HDAC (approved for use in Cutaneous T-cell lymphoma); Ziv-aflibercept (Zaltrap) targeting PIGF, VEGFA/B (approved for use in Colorectal cancer); and the like.

Biological response modifiers suitable for use in connection with the methods of the present disclosure include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; ( 4) apoptosis receptor agonists; (5) interleukin-2; (6) interferon-a.; (7) interferon -γ; (8) colony-stimulating factors; (9) inhibitors of angiogenesis; and (10) antagonists of tumor necrosis factor.

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan®), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17a-ethinylestradiol; diethylstilbestrol, testosterone, fluoxymesterone, dromostanolone propionate, testolactone, methylprednisolone, methyltestosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL®, TAXOTERE® (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, paclitaxel-xylose, or paclitaxel-albumin).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Useful immunotherapies include: anti-PD-1/PD-L1 immunotherapies, and/or other immunotherapy targets, such as e.g., immune check point markers, such as CTLA-4, LAG-3 and TIM-3, that may be targeted in treatment methods. Anti-PD-1/PD-L1 immunotherapies which include but are not limited to e.g., those therapies that include administering to a subject an effective amount of one or more anti-PD-1/PD-L1 therapeutic antagonists where such antagonists include but are not limited to e.g., OPDIVO® (nivolumab), KEYTRUDA® (pembrolizumab), Tecentriq™ (atezolizumab), durvalumab (MEDI4736), avelumab (MSB0010718C), BMS-936559 (MDX-1105), CA-170, BMS-202, BMS-8, BMS-37, BMS-242 and the like.

CTLA-4, also known as CD152, binds to CD80 and CD86. Antibodies against CTLA-4 have been approved for treating some cancer types. The co-inhibitory effect of CTLA-4 with other immunotherapies make CTLA-4 a good candidate for use in combination with other immunotherapies to treat certain cancers. TIM-3 may also be targeted for immunotherapy for several cancer types.

LAG-3 is in clinical trials for treating cancers. Anti-LAG-3 immunotherapies include those that employ antagonist LAG-3 antibodies that can both activate T effector cells (by downregulating the LAG-3 inhibiting signal into pre-activated LAG-3+ cells) and inhibit induced (i.e. antigen-specific) Treg suppressive activity. Useful LAG-3 antagonistic antibodies include relatlimab (BMS-986016; developed by Bristol-Myers Squibb), IMP701 (developed by Immutep), TSR-033 (anti-LAG-3 mAb; developed by TESARO, Inc.), and the like.

Immunotherapies also include T cell-based immunotherapies such as e.g., adoptive cell therapy (ACT) and chimeric antigen receptor (CAR) T cell therapies. For example, a subject may be administered a population of CAR T cells engineered to target an antigen expressed by the subject’s cancer. A T cell-based therapy may involve, in some instances, obtaining a cellular sample from a subject, such as a blood sample or a tumor biopsy, and culturing immune cells from the sample ex vivo, with or without genetic modification of the cultured immune cells. As an example, immune cells may be obtained from a subject, cultured ex vivo and modified with a CAR specific for an antigen expressed by the cancer to produce a population of CAR T cells. Then, the CAR T cells may be reintroduced into the subject to target the cancer. T cell-based immunotherapies may be configured in various ways, e.g., by targeting various antigens, by collecting/culturing various cell types, etc., depending on a particular cancer to be treated. In addition, T cell-based immunotherapies may be administered systemically, e.g., by intravenous injection, or locally, e.g., by infusion (e.g., intraperitoneal infusion, pleural catheter infusion, etc.), direct injection, and the like.

In some instances, a method of treatment described herein may include administering to a subject one or more inhibitors of a multidrug resistance transporter, including but not limited to e.g., a multidrug resistance transporter other than ABCG2. Useful inhibitors of multidrug resistance transporters include e.g., tyrosine kinase inhibitors, natural products, microRNAs, and small molecule inhibitors. Inhibitors of multidrug resistance transporters include ABC transporter inhibitors.

Individuals suitable for treatment using a method of the present disclosure include an individual having a cancer; an individual diagnosed as having a cancer; an individual being treated for a cancer with chemotherapy, radiation therapy, antibody therapy, surgery, etc.); an individual who has been treated for a cancer (e.g., with one or more of chemotherapy, radiation therapy, antibody therapy, surgery, etc.), and who has failed to respond to the treatment; an individual who has been treated for a cancer (e.g., with one or more of chemotherapy, radiation therapy, antibody therapy, surgery, etc.), and who initially responded to the treatment but who subsequently relapsed, i.e., the cancer recurred.

The methods of the present disclosure may be employed to target and treat a variety of cancers, including e.g., primary cancer, secondary cancers, re-growing cancers, recurrent cancers, refractory cancers and the like. For example, in some instances, the methods of the present disclosure may be employed as an initial treatment of a primary cancer identified in a subject. In some instances, the methods of the present disclosure may be employed as a non-primary (e.g., secondary or later) treatment, e.g., in a subject with a cancer that is refractory to a prior treatment, in a subject with a cancer that is re-growing following a prior treatment, in a subject with a mixed response to a prior treatment (e.g., a positive response to at least one tumor in the subject and a negative or neutral response to at least a second tumor in the subject), and the like.

In some instances, the methods of the present disclosure may be employed to treat a subject with a drug resistant cancer, such as a multi-drug resistant cancer. Multidrug resistance (MDR) is the mechanism by which many cancers develop resistance to chemotherapy drugs, resulting in minimal cell death and the expansion of drug-resistant tumors. MDR cancers may involve one or more resistance mechanisms including but not limited to e.g., increased expression of efflux pumps, decreased absorption of drug, inhibition of cell death or apoptosis, modulating drug metabolism, and the like. In some instances, the methods of the present disclosure may prevent, reverse or circumvent MDR.

In some instances, methods of the present disclosure may include treating a subject having a cancer that is resistant to a first agent with an effective amount of a subject multi-specific antibody described herein in combination with a second agent that is different from the first agent. For example, in some instances, cancer of a subject may be resistant to a first chemotherapeutic and the subject may be treated by administering an effective amount of a subject multi-specific antibody as described herein in combination with a second chemotherapeutic that is different from the first. Various combinations of first and second chemotherapeutics may be employed depending on e.g., the type of cancer to be treated, the likelihood of developing resistance, etc.

Numerous cancers are known to develop drug resistance. For this and other reasons the methods of the present disclosure may find use in treating various cancers including but not limited to, e.g., Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, etc.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenström, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sézary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, and the like.

The methods of treating described herein may, in some instances, be performed in a subject that has previously undergone one or more conventional treatments. For example, in the case of oncology, the methods described herein may, in some instances, be performed following a conventional cancer therapy including but not limited to e.g., conventional chemotherapy, conventional radiation therapy, conventional immunotherapy, surgery, etc. In some instances, the methods described herein may be used when a subject has not responded to or is refractory to a conventional therapy. In some instances, the methods described herein may be used when a subject has responded to a conventional therapy.

In some instances, the method of the present disclosure may be employed to target, treat or clear a subject for minimal residual disease (MRD) remaining after a prior cancer therapy. Targeting, treating and/or clearance of MRD may be pursued using the instant methods whether the MRD is or has been determined to be refractory to the prior treatment or not. In some instances, a method of the present disclosure may be employed to target, treat and/or clear a subject of MRD following a determination that the MRD is refractory to a prior treatment or one or more available treatment options other than those employing the herein described multi-specific antibodies.

In some instances, the instant methods may be employed prophylactically for surveillance. For example, a subject in need thereof may be administered a treatment involving one or more of the herein described multi-specific antibodies when the subject does not have detectable disease but is at risk of developing a recurrent cancer, including e.g., a drug resistant cancer. In some instances, a prophylactic approach may be employed when a subject is at particularly high risk of developing a primary cancer that would be predicted to be drug resistant or expected to become drug resistant. In some instances, a prophylactic approach may be employed when a subject has been previously treated for a cancer and is at risk of reoccurrence or development of drug resistance.

In some instances, methods of the present disclosure may involve analyzing a cancer for expression of one or more markers or therapeutic targets. For example, in some instances, methods may involve analyzing a sample of a cancer from a subject to determine whether the cancer expresses ABCG2 above a predetermined threshold, a TAA (e.g., CD47, erbB2, or EGFR) above a predetermined threshold, or both.

In some instances, whether a subject is treated with a multi-specific antibody of the present disclosure may depend on the results of the TAA and/or ABCG2 testing. For example, in some instances, if a cancer expresses the TAA at or above a predetermined threshold then the subject may be treated with a multi-specific antibody of the present disclosure, and if the cancer expresses the TAA below the predetermined threshold then the subject may not be treated with the multi-specific antibody, e.g., the subject may be treated with a conventional therapy for the relevant cancer without the subject multi-specific antibody. In some instances, if a cancer expresses ABCG2 at or above a predetermined threshold then the subject may be treated with a multi-specific antibody of the present disclosure, and if the cancer expresses ABCG2 below the predetermined threshold then the subject may not be treated with the multi-specific antibody, e.g., the subject may be treated with a conventional therapy for the relevant cancer without the subject multi-specific antibody. In some instances, if a cancer expresses both the TAA and ABCG2 at or above predetermined thresholds then the subject may be treated with a multi-specific antibody of the present disclosure, and if the cancer expresses the TAA and ABCG2 below the predetermined thresholds then the subject may not be treated with the multi-specific antibody, e.g., the subject may be treated with a conventional therapy for the relevant cancer without the subject multi-specific antibody.

Any convenient assay may be employed for analyzing ABCG2 and/or TAA levels, including but not limited to e.g., flow cytometry, nucleic acid-based assays (e.g., amplification, sequencing, etc.), cell cytometry, immunohistochemistry, and the like. Any convenient biological sample may be employed, including but not limited to e.g., cancer biopsy samples. Useful predetermined thresholds for assessing expression of one or more markers and/or targets may be determined by any convenient and appropriate method, including comparison of the measured level of expression to a corresponding control. For example, in some instances, a useful predetermined threshold for the level of ABCG2 and/or TAA assayed in a sample may correspond to a level of ABCG2 and/or TAA as measured in a reference cell, such as a healthy/normal cell. The TAA may be CD47, erbB2, EGFR, or PD-L1.

### Methods of Making

As summarized above, methods of the present disclosure also include methods or making and/or identifying multi-specific antibodies as described herein. A subject antibody can be produced by any known method, e.g., conventional synthetic methods for protein synthesis; recombinant DNA methods; etc.

Where a subject antibody is a single chain polypeptide, it can synthesized using standard chemical peptide synthesis techniques. Where a polypeptide is chemically synthesized, the synthesis may proceed via liquid-phase or solid-phase. Solid phase polypeptide synthesis (SPPS), in which the C-terminal amino acid of the sequence is attached to an insoluble support followed by sequential addition of the remaining amino acids in the sequence, is an example of a suitable method for the chemical synthesis of a subject antibody. Various forms of SPPS, such as Fmoc and Boc, are available for synthesizing a subject antibody. Techniques for solid phase synthesis are described by Barany and Merrifield, Solid-Phase Peptide Synthesis; pp. 3-284 in The Peptides: Analysis, Synthesis, Biology. Vol. 2: Special Methods in Peptide Synthesis, Part A., Merrifield, et al. J. Am. Chem. Soc., 85: 2149-2156 (1963); Stewart et al., Solid Phase Peptide Synthesis, 2nd ed. Pierce Chem. Co., Rockford, III. (1984); and Ganesan A. 2006 Mini Rev. Med Chem. 6:3-10 and Camarero JA et al. 2005 Protein Pept Lett. 12:723-8. Briefly, small insoluble, porous beads are treated with functional units on which peptide chains are built. After repeated cycling of coupling/deprotection, the free N-terminal amine of a solid-phase attached is coupled to a single N-protected amino acid unit. This unit is then deprotected, revealing a new N-terminal amine to which a further amino acid may be attached. The peptide remains immobilized on the solid-phase and undergoes a filtration process before being cleaved off.

Standard recombinant methods can be used for production of a subject antibody. For example, nucleic acids encoding light and heavy chain variable regions, optionally linked to constant regions, are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Expression control sequences include, but are not limited to, promoters (e.g., naturally-associated or heterologous promoters), signal sequences, enhancer elements, and transcription termination sequences. The expression control sequences can be eukaryotic promoter systems in vectors capable of transforming or transfecting eukaryotic host cells (e.g., COS or CHO cells). Once the vector has been incorporated into the appropriate host, the host is maintained under conditions suitable for high level expression of the nucleotide sequences, and the collection and purification of the antibodies.

Because of the degeneracy of the code, a variety of nucleic acid sequences can encode each immunoglobulin amino acid sequence. The desired nucleic acid sequences can be produced by de novo solid-phase DNA synthesis or by polymerase chain reaction (PCR) mutagenesis of an earlier prepared variant of the desired polynucleotide. Oligonucleotide-mediated mutagenesis is an example of a suitable method for preparing substitution, deletion and insertion variants of target polypeptide DNA. See Adelman et al., DNA 2:183 (1983). Briefly, the target polypeptide DNA is altered by hybridizing an oligonucleotide encoding the desired mutation to a single-stranded DNA template. After hybridization, a DNA polymerase is used to synthesize an entire second complementary strand of the template that incorporates the oligonucleotide primer, and encodes the selected alteration in the target polypeptide DNA.

Suitable expression vectors are typically replicable in the host organisms either as episomes or as an integral part of the host chromosomal DNA. Commonly, expression vectors contain selection markers (e.g., ampicillin-resistance, hygromycin-resistance, tetracycline resistance, kanamycin resistance or neomycin resistance) to permit detection of those cells transformed with the desired DNA sequences.

Escherichia coli is an example of a prokaryotic host cell that can be used for cloning a subject antibody-encoding polynucleotide. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Other microbes, such as yeast, are also useful for expression. Saccharomyces (e.g., S. cerevisiae) and Pichia are examples of suitable yeast host cells, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

In addition to microorganisms, mammalian cells (e.g., mammalian cells grown in in vitro cell culture) can also be used to express and produce the polypeptides of the present invention (e.g., polynucleotides encoding immunoglobulins or fragments thereof). See Winnacker, From Genes to Clones, VCH Publishers, N.Y., N.Y. (1987). Suitable mammalian host cells include CHO cell lines, various Cos cell lines, HeLa cells, myeloma cell lines, and transformed B-cells or hybridomas. Expression vectors for these cells can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Examples of suitable expression control sequences are promoters derived from immunoglobulin genes, SV40, adenovirus, bovine papilloma virus, cytomegalovirus and the like. See Co et al., J. Immunol. 148:1149 (1992).

Once synthesized (either chemically or recombinantly), the whole antibodies, their dimers, individual light and heavy chains, or other forms of a subject antibody (e.g., scFv, etc.) can be purified according to standard procedures of the art, including ammonium sulfate precipitation, affinity columns, column chromatography, high performance liquid chromatography (HPLC) purification, gel electrophoresis, and the like (see generally Scopes, Protein Purification (Springer-Verlag, N.Y., (1982)). A subject antibody can be substantially pure, e.g., at least about 80% to 85% pure, at least about 85% to 90% pure, at least about 90% to 95% pure, or 98% to 99%, or more, pure, e.g., free from contaminants such as cell debris, macromolecules other than a subject antibody, etc.

In some embodiments, method of generating a multi-specific antibody of the present disclosure may include producing candidate antibodies and screening for activity. Such methods may generate a multi-specific antibody that specifically binds a cell expressing both ABCG2 and TAA through the use of a series of steps. Steps of such methods may include: producing a multi-specific antibody or a plurality of antibodies that each include or are expected to include a ABCG2-binding domain and a TAA-binding domain; contacting a first test cell expressing ABCG2 and TAA with the multi-specific antibody or plurality of antibodies; contacting a second cell expressing either ABCG2 or TAA with the multi-specific antibody or plurality of antibodies; comparing the binding of the multi-specific antibody, or the antibodies of the plurality, to the first cell with the binding of the multi-specific antibody to the second cell to determine a binding-specificity ratio; and identifying the multi-specific antibody, or one or more of the antibodies of the plurality, as specific for the cell expressing both ABCG2 and TAA when the ratio is above a predetermined threshold. Where such a threshold for comparative binding is employed, the threshold may vary and may range from 1.5:1 or more, including but not limited to e.g., 2:1, 3:1, 4:1, 5:1, 6:1, 7:1, 8:1, 9:1, 10:1, 20:1, 50:1, 100:1, etc.

Various cells may be used in such methods, including but not limited to e.g., the cells described herein. In some instances, the binding of the antibody to both ABCG2-only expressing cells and TAA-only expressing cells may be performed. For example, in some instances, the method may include, relative to the steps describe above, where the second cell expresses ABCG2 and not TAA and the method further comprises contacting a third cell expressing TAA but not ABCG2 with the multi-specific antibody.

In some instances, such methods may employ one or more controls, including but not limited to e.g., control cells, control reagents, and the like. Useful control cells include those that have a known expression or known lack of expression of one or more relevant genes or proteins. Useful control reagents may include but are not limited to e.g., control antibodies such as but not limited to e.g., monospecific antibodies to known targets. For example, in some instances, such methods of the present disclosure may further include contacting the first cell, the second cell, and/or the third cell with a control antibody selected from: a monospecific anti-ABCG2 antibody and a monospecific anti-TAA antibody. Depending on the particular method used, various other or additional controls, as appropriate, may be employed.

### Kits

Aspects of the present disclosure also include kits. The kits may include, e.g., any combination of the multi-specific antibodies, reagents, compositions, formulations, cells, nucleic acids, expression vectors, or the like, described herein. A subject kit can include one or more of: a subject multi-specific antibody, a nucleic acid encoding the same, or a cell comprising a subject multi-specific nucleic acid. Kits may be configured for various purposes, including e.g., treatment kits (e.g., where a kit may include a multi-specific antibody and e.g., one or more additional active agents, such as a chemotherapeutic), kits for producing antibodies, kits for screening antibodies, and the like.

Optional components of the kit will vary and may, e.g., include: a buffer; a protease inhibitor; etc. Where a subject kit comprises a subject nucleic acid, the nucleic acid may also have restrictions sites, multiple cloning sites, primer sites, etc. The various components of the kit may be present in separate containers or certain compatible components may be pre-combined into a single container, as desired.

In addition to above-mentioned components, a subject kit can include instructions for using the components of the kit to practice a subject method. The instructions for practicing a subject method are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. compact disc-read only memory (CD-ROM), digital versatile disk (DVD), diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXEMPLARY NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1: Generation of Bispecific mAbs That Sensitize Cells to Topotecan

### Materials and Methods

### Generation of a Stable ABCG2 Overexpressing (Ox) Cell Lines

In order to characterize both binding and in vitro efficacy, a cell line that stably over-expressed ABCG2 was developed. Adherent 293T naïve cells obtained from American Type Culture Collection (ATCC) were utilized. As characterized by flow cytometry using a commercially available ABCG2 antibody (R&D System, clone 5D3), this cell line expresses ABCG2 endogenously at a low to moderate degree on the cell surface. 293T naïve cells, 3T3 cells or C6 cells (QZ) were transfected with ABCG2 using Polyplus PEIpro reagent. Three days after transfection, cells were put under selection using a Hygromycin B solution (Millipore Sigma). Fourteen days after continuous Hygromycin B selection 293T cells were evaluated for ABCG2 cell surface expression. To ensure non-transfected cells would not expand in future cultures, a bulk sort using fluorescent activated cell sorting (FACS) of ABCG2 positive 293T cells was performed using a FACSArial (BD Biosciences). The bulk sorted 293T ABCG2 over-expressing cells were expanded and ABCG2 over-expression was subsequently re-confirmed.

### Cell Culture Techniques and Antibody Production

Standard cell culture techniques are used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

293 cells were used for transient production of bispecific mAbs. Different antibody constructs were expressed using polymer-based co-transfection of Expi293 cells (A14527, ThermoFisher). Cells were grown in suspension with the mammalian expression vectors following the manufacturer’s recommendations.

For preparation of bispecific antibody molecules, cells were transfected with the corresponding expression vectors in a 1:1:4 ratio (heavy chain KK: heavy chain DD: light chain). For standard antibody expression a 1:2 ratio (heavy chain: light chain) was used.

About six days after transfection the cells were harvested by centrifugation. In detail, 1 µg of total encoding DNA per 1ml of transfected culture was diluted into of Opti-MEM® medium (Life Technologies), and incubated with Expifectamine reagent (Life Technologies) in the same medium for 20 min. The mixture was then added into the Expi293® cells growing in suspension in Expi293® Expression medium (Life Technologies) at 2.5 million cells/ml at 37° C. with and overlay of 8% of CO₂ in air. After 6 days, the medium containing the antibody construct was harvested by centrifugation.

### Construction of the Human-Mouse Sequences of the Molecules as Tested (Human Fc, Mouse Fvs or Humanized Fvs)

Expression Vectors: For the generation of the antibody expression vectors, the variable regions of heavy and light chain DNA sequences were subcloned in frame with either the human IgG1 constant heavy chain or the human IgG1 kappa constant light chain pre-inserted into the respective generic recipient expression vector optimized for expression in mammalian cell lines. The genes to be expressed were cloned into the pCI-neo Mammalian Expression Vector (Promega) that uses the full-length human cytomegalovirus (CMV) immediate-early promoter for high level gene expression. The different antibody chains were cloned into different vectors.

The N-terminal signal sequences from mouse IgG heavy chain and kappa light chain were used for the secreted expression of the heavy and light chain, respectively. The signal peptide was cleaved during expression, leaving intact N-terminus. In the Fab constructs, the C-terminus of the CH1 IgG1 constant region was fused with a 6× His tag to facilitate purification.

For the generation of bispecific antibody vectors, the IgG1 derived bispecific molecules include at least of two antigen binding moieties capable of binding specifically to two distinct targets: TAA and ABCG2. The antigen binding moieties are Fab fragments composed of a heavy and a light chain, each including a variable and a constant region. A common light chain was identified that was able to pair and effect acceptable binding both as Fab anti-TAA and Fab anti-ABCG2 (aABCG2); its use enabled the avoidance of LC mispairing. Bispecific constructs were made based on electrostatic steering effects, (see e.g., Gunasekeran et al, (2010) Journal of Biological Chemistry 285, 19637-19646; the disclosure of which is incorporated herein by reference in its entirety). Briefly, the polypeptide chains or half antibodies against the targets are assembled as a bispecific antibody through charge pair substitutions at the CH3 domain: one heavy chain contained K392D and K409D substitutions (“DD”) and the other contained E356K and D399K substitutions (“KK”).

Variable heavy and light chain fragments from mouse hybridoma sequences are available and were cloned into the same background of leader sequence and constant region.

Cell Binding Assays. Antibody binding to cells was evaluated by flow cytometry. 293T cells stably transfected to express human or cynomolgus ABCG2 (293T_ABCG2_OX) or KT9 (293T-KT9OX) were washed once in flow cytometry buffer (PBS + 2% FBS + 0.02% sodium azide), resuspended at 2 × 10^6 cells/mL in flow cytometry buffer, and dispensed into 96-well microtiter plates at 0.1 mL/well. Recombinant antibodies were added to cells at 5 ug/mL for initial binding confirmation, or serially diluted from 100 ug/mL in flow cytometry buffer. After incubating cells on ice for 30 min, cells were washed twice with flow cytometry buffer. Bound antibody was detected with PE-labeled F(ab′)₂ fragment goat anti-human IgG (Jackson ImmunoResearch) and evaluated on an Attune NxT flow cytometer. EC50 is calculated to be the concentration of antibody that gives half maximal response.

Cytotoxicity Assays. The effect of antibodies on topotecan cytotoxicity was evaluated on 293T_ABCG2_OX cells, 293T cells stably transfected to express ABCG2. Cells were plated in 0.05 mL of Assay Media (DMEM +10% FBS) at 5000 cells/well in white, flat bottom 96-well tissue culture plates. Topotecan was prepared at 2X final assay concentration by serial dilution from 200 uM in assay media containing test antibodies or control antibodies at 100 ug/mL (2X final concentration), or Fumitremorgin C (FTC), a small molecule ABCG2 inhibitor at (2X final concentration). An equivalent volume (0.05 mL) of the topotecan/antibody mixture was added to the 293T_ABCG2_OX cells in 96-well plates. The plates were then incubated at 37° C., in 5% CO₂. After about 72-96 hr plates were equilibrated to room temperature and cell viability assessed using Promega® CellTiter-Glo® Luminescent Cell Viability Assay according to the manufacturer’s recommended protocol. Luminescence was measured on a Molecular Devices® FlexStation® 3 Multi-Mode Microplate Reader and data analyzed using GraphPad Prism 8.0 software. Half maximal inhibitory concentration (IC50) is the concentration of drug (topotecan or other chemotherapy cytotoxic agent) where the response (cell growth) is reduced by 50%.

### Xenograft Studies

Materials:
Cells: HT1376 (ATCC CRL-1472) human urinary bladder carcinoma cell lines.
Mice: Sixty-five 5-6-weeks-old female SCID-Biege mice (Charles River).
Reagents: G2KT9 anti-ABCG2 × anti-CD4 BsAb produced as described above, Human Isotype IgG1 (Bioxcell), topotecan.

Methods:
Cell culture: HT1376 cells were maintained in RPMI medium supplemented with 10% FBS and 1% penicillin and 1% streptomycin at 37° C., 5% CO₂. Cell lines used were authentic and confirmed to be mycoplasma negative.

Inoculation-2 X 10⁶ cells diluted in PBS:Matrigel (1:1) were subcutaneously injected using a 27G insulin syringe into fifty anesthetized 5-6-week-old female SCID-Biege mice under sterile conditions.

### Sequences

Anti-ABCG2 5D3 antibody variable heavy (VH) chain sequence is as follows:

QVQLQESGPGLVKPSQSLSLTCTVTGFSITSDYAWNWIRQFPGKKLEWMGY
INFDGGTTYNPSLRGRISITRDTSKNQFFLQLRSVTPEDTATYYCATFYGA
KGTLDYWGQGTSVTVSS (SEQ ID NO:5)

Humanized versions (v1 and v2) of 5D3 VH chain were created.

Humanized 5D3 VHv1 sequence is as follows:

EVQLQESGPGLVKPSETLSLTCTVSGFSITSDYAWNWIRQPPGKGLEWMGY
INFDGGTTYNPSLRGRITISRDTSKNQFSLKLSSVTAADTAVYYCATFYGA
KGTLDYWGQGTLVTVSS (SEQ ID NO:6)

Humanized 5D3 VHv2 sequence is as follows:

EVQLQESGPGLVKPSETLSLTCTVSGFSITSDYAWNWIRQPPGKGLEWIGY
INFDGGTTYNPSLRGRVTISRDTSKNQFSLKLSSVTAADTAVYYCATFYGA
KGTLDYWGQGTLVTVSS (SEQ ID NO:7)

Anti-ABCG2 5D3 antibody variable light chain sequence is as follows:

DIVLTQSPSSFSVSLGDRVTISCKASGYILNRLAWYQQKPGNAPRLLISG
ATSLETGFPSRFSGTGSGKDYTLSISSLQTEDVGTYYCQQYWSTPWTFGG
GTKLEIR (SEQ ID NO:1)

Humanized version (v1) of 5D3 VL chain was created.

Humanized 5D3 VLv1 sequence is as follows:

DIQLTQSPSSLSASVGDRVTITCKASGYILNRLAWYQQKPGKAPKLLISG
ATSLETGFPSRFSGSGSGKDYTLTISSLQPEDFATYYCQQYWSTPWTFGG
GTKLEIK (SEQ ID NO:59)

Sequences encoding CD47 are available: Homo sapiens CD47 molecule (CD47), transcript variant 1, mRNA NCBI Reference Sequence: NM_001777.3 Anti-CD47 5F9 antibody variable heavy chain sequence is as follows:

QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSS (SEQ ID NO:8).

Anti-CD47 5F9 antibody variable light chain sequence is as follows:

DIVMTQSPLSLPVTPGEPASISCRSSQSIVYSNGNTYLGWYLQKPGQSPQ
LLIYKVSNRFSGVPDRFSGSGSGTDFTLKISRVEADVGVYYCFQGSHVPY
TFGQGTKLEIK (SEQ ID NO:60).

Anti-erbB2 antibody, Pertuzumab, heavy chain and light chain sequence are as follows: Pertuzumab heavy chain sequence:

EVQLVESGGGLVQPGGSLRLSCAASGFTFTDYTMDWVRQAPGKGLEWVAD VNPNSGGSIYNQRFKGRFTLSVDRSKNTLYLQMNSLRAEDTAVYYCARNL GPSFYFDYWGQGTLVTVSS ASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPG
(SEQ ID NO:16)

VH chain is indicated in bold and is underlined.

Pertuzumab light chain sequence:

DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC (SEQ ID NO:61)

Anti-erbB1 antibody, Necitumumab, heavy chain and light chain sequence are as follows: Necitumumab heavy chain sequence:

QVQLQESGPGLVKPSQTLSLTCTVSGGSISSGDYYWSWIRQPPGKGLEWI GYIYYSGSTDYNPSLKSRVTMSVDTSKNQFSLKVNSVTAADTAVYYCARV SIFGVGTFDYWGQGTLVTVSS ASTKGPSVLPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
K (SEQ ID NO:17)

VH chain is indicated in bold and is underlined.

Necitumumab light chain sequence:

EIVMTQSPATLSLSPGERATLSCRASQSVSSYLAWYQQKPGQAPRLLIYD ASNRATGIPARFSGSGSGTDFTLTISSLEPEDFAVYYCHQYGSTPLTFGG GTKAEIK RTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC (SEQ ID NO:62)

VL chain is indicated in bold and is underlined.

Anti-PD-L1 antibody, Atezolizmumab, variable heavy chain sequence is as follows:

EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS (SEQ ID NO:27).

### Results

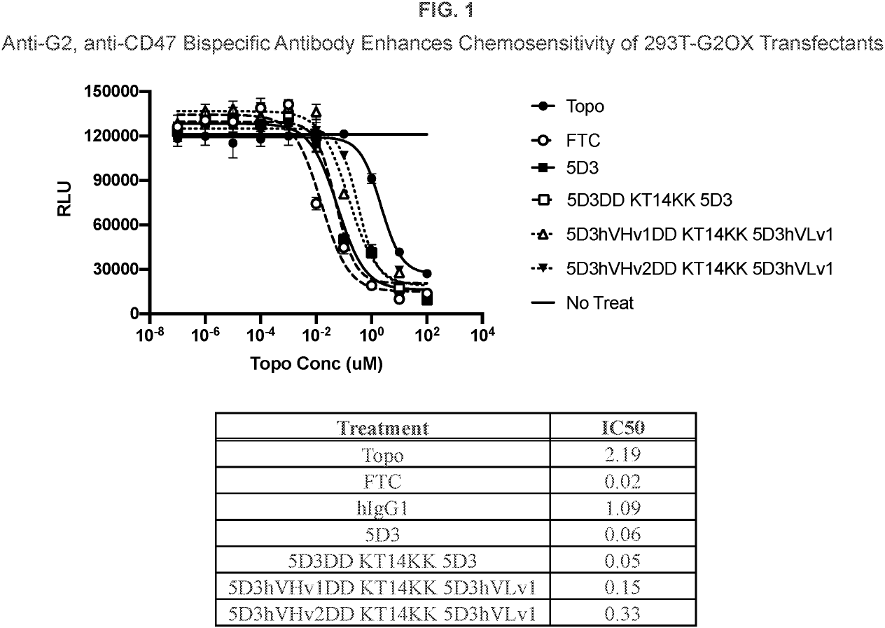

FIG. 1 shows that 293T cells overexpressing ABCG2 have increased susceptibility to topotecan in the presence of the anti-ABCG2 antibody 5D3 as well as a bispecific antibody that binds to ABCG2 and CD47. The bispecific antibody 5D3DDKT14KK5D3 includes heavy chain (HC) and light chain (LC) from the anti-ABCG2 antibody 5D3 and a HC from the anti-CD47 antibody, 5F9. The bispecific antibody 5D3hVHv1DDKT14KK5D3hVLv1 includes HC and LC from the anti-ABCG2 antibody 5D3 and a HC from the anti-CD47 antibody, 5F9. The bispecific antibody 5D3hVHv2DDKT14KK5D3hVLv1 includes HC and LC from the anti-ABCG2 antibody 5D3 and a HC from the anti-CD47 antibody, 5F9. hVHv1 and hVHv2 refer to humanized variable heavy chain version 1 and version 2, respectively, of the 5D3 HC. hVLv1 refers to humanized variable light chain version 1 of the 5D3 LC. KT14 refers to CD47. DD and KK refer to charged pair substitutions that enhance pairing between 5D3 and 5F9 HCs. IC50 values are in nM.

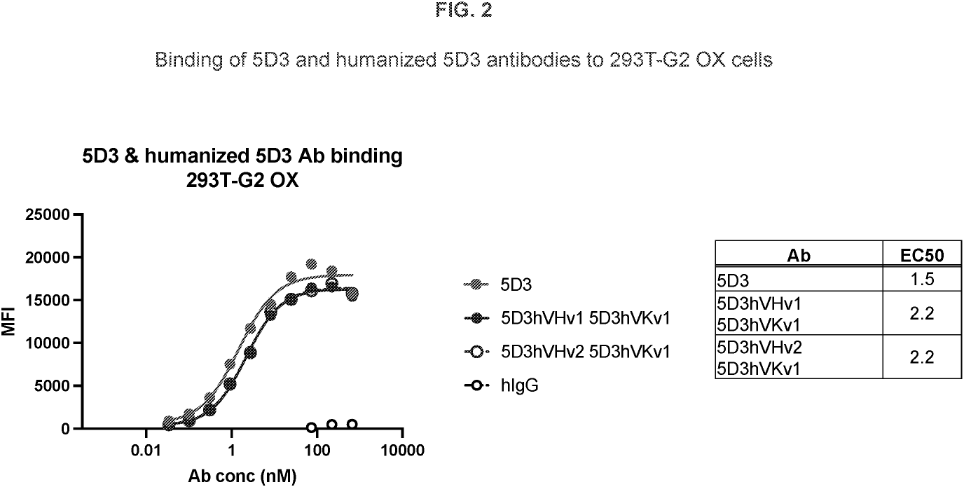

FIG. 2 shows the binding of 5D3 and humanized 5D3 antibodies to 293T cells stably transfected to express human ABCG2 (293T-G2 OX) along with the corresponding EC50 values. The results show that humanization does not significantly interfere with the potency of the 5D3 antibody.

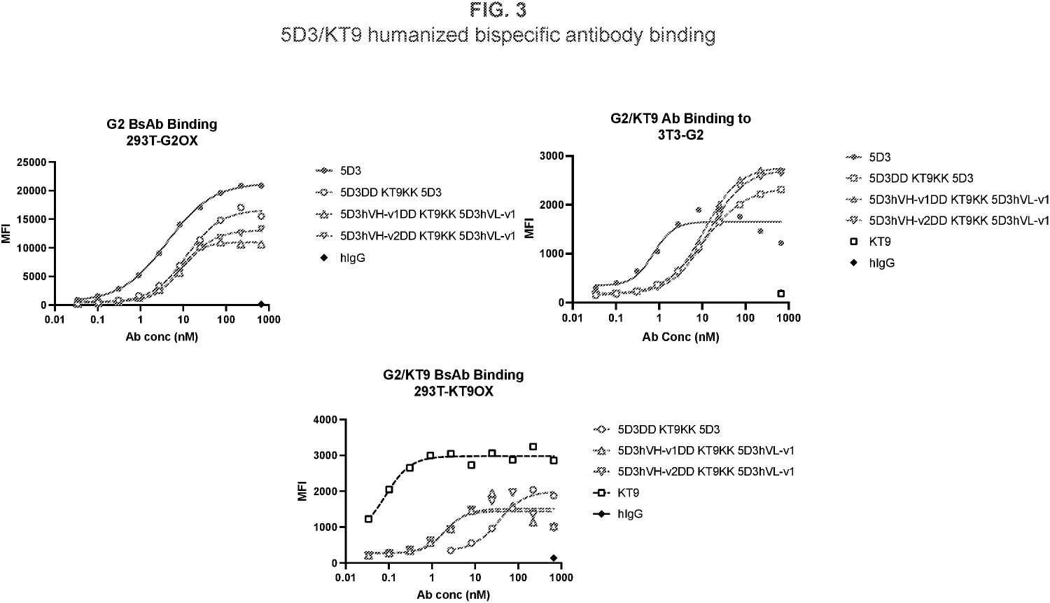

FIG. 3 shows the binding of various humanized ABCG2/KT9 bispecific antibodies to 3T3 cells stably transfected to express ABCG2 (3T3-G2), 293T cells stably transfected to express human ABCG2 (293T_ABCG2_OX), and 293T cells stably transfected to express human KT9 (293T-KT9OX). KT9 is atezolizumab, an anti-PD-L1 antibody, and 5D3 is an anti-ABCG2 antibody. The humanized bispecific antibodies 5D3hVH-v1DD KT9KK 5D3hVL-v1 and 5D3hVH-v2DD KT9KK 5D3hVL-v1 include HC and LC from the anti-ABCG2 antibody 5D3 and a HC from the anti-PD-L1 antibody, KT9. The results show that the humanized bispecific antibodies retain the ability to bind their targets, ABCG2 and PD-L1, respectively.

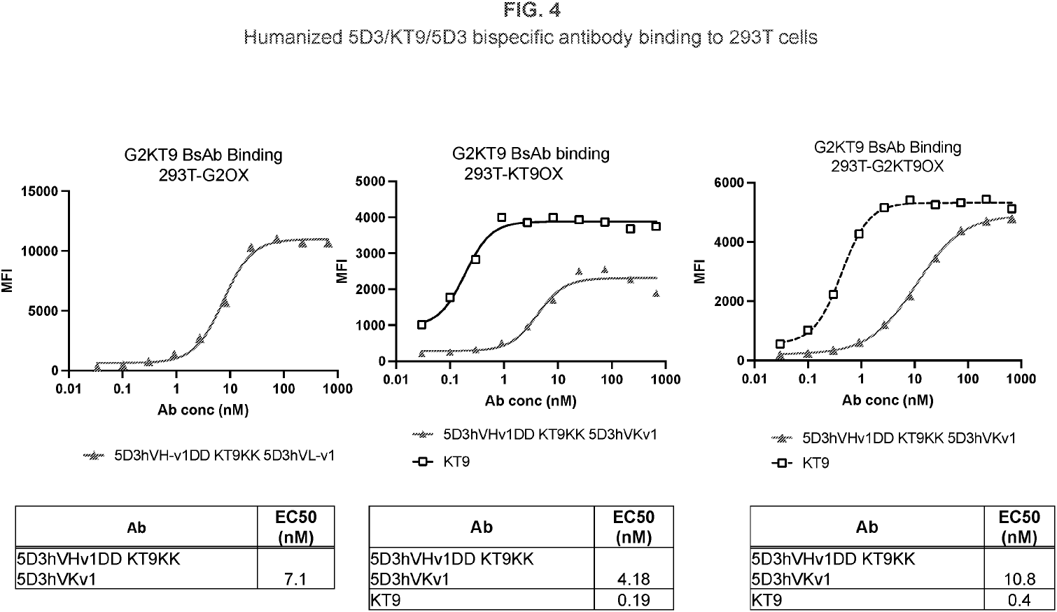

FIG. 4 shows the binding of humanized 5D3/KT9/5D3 bispecific antibodies to 293T cells stably transfected with ABCG2 (293T-G2OX), KT9 (293T-KT9OX) and both ABCG2 and KT9 (293T-G2KT9OX), along with the corresponding EC50 binding affinities. The results show that the tested humanized antibodies retain the ability to bind their targets.

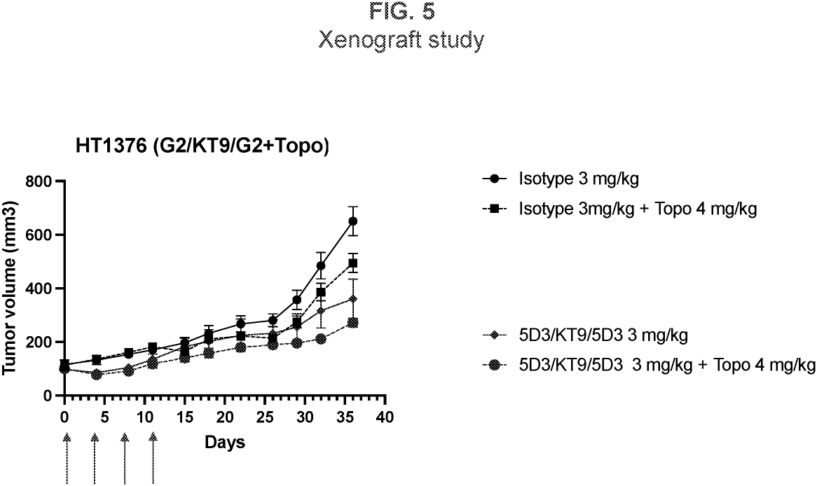

FIG. 5 shows the results of a xenograft study where the cytotoxic activity of an ABCG2/PD-L1 bispecific antibody (5D3/KT9) alone or in combination with topotecan, was tested in the HT1376 (ATCC CRL-1472) human urinary bladder epithelial carcinoma cell line. The bispecific antibody is shown to be efficacious both as a single agent and in combination with topotecan.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. §112(f) or 35 U.S.C. §112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. §112(6) is not invoked.

What is claimed is:

1. A bispecific antibody molecule that binds ATP Binding Cassette Subfamily G Member 2 (ABCG2) and a tumor associated antigen (TAA), the antibody molecule comprising two identical variable light (VL) chains, a first variable heavy (VH) chain, and a second VH chain,
wherein the VL chains each comprise an antigen-binding site for ABCG2, the first VH chain comprises an antigen-binding site for ABCG2, and the second VH chain comprises an antigen-binding site for the TAA, and wherein the second VH chain binds the TAA when paired with one of the VL chains,
wherein the bispecific antibody binds to cancer cells expressing both ABCG2 and the TAA while showing reduced binding to non-cancer cells expressing ABCG2 and/or the TAA.

2. The bispecific antibody molecule according to claim 1, wherein the antigen-binding site of the two VL chains comprises light chain CDRs 1-3 (LCDRs 1-3) of a VL chain having the sequence:
DIVLTQSPSSFSVSLGDRVTISCKASGYILNRLAWYQQKPGNAPRLLISG
ATSLETGFPSRFSGTGSGKDYTLSISSLQTEDVGTYYCQQYWSTPWTFGG
GTKLEIR (SEQ ID NO:1).

3. The bispecific antibody molecule according to claim 2, wherein the two VL chains comprise LCDRs 1-3, wherein LCDR1 comprises the sequence KASGYILNRLA (SEQ ID NO:2); LCDR2 comprises the sequence GATSLET (SEQ ID NO:3) and LCDR3 comprises the sequence QQYWSTPWT (SEQ ID NO:4).

4. The bispecific antibody molecule according to any one of claims 1 to 3, wherein the two VL chains comprise the sequence or an amino acid sequence at least 90%, at least 95%, or at least 99% identical to the sequence:
DIVLTQSPSSFSVSLGDRVTISCKASGYILNRLAWYQQKPGNAPRLLISG
ATSLETGFPSRFSGTGSGKDYTLSISSLQTEDVGTYYCQQYWSTPWTFGG
GTKLEIR (SEQ ID NO:1);
or
DIQLTQSPSSLSASVGDRVTITCKASGYILNRLAWYQQKPGKAPKLLISG
ATSLETGFPSRFSGSGSGKDYTLTISSLQPEDFATYYCQQYWSTPWTFGG
GTKLEIK (SEQ ID NO:59).

5. The bispecific antibody molecule according to any one of the preceding claims, wherein the antigen binding site of first VH chain comprises heavy chain CDRs 1-3 (HCDRs 1-3) of a VH chain having the sequence:
QVQLQESGPGLVKPSQSLSLTCTVTGFSITSDYAWNWIRQFPGKKLEWMG
YINFDGGTTYNPSLRGRISITRDTSKNQFFLQLRSVTPEDTATYYCATFY
GAKGTLDYWGQGTSVTVSS (SEQ ID NO:5).

6. The bispecific antibody molecule according to any one of claims 1-4, wherein the antigen binding site of first VH chain comprises heavy chain CDRs 1-3 (HCDRs 1-3), wherein:
(i) the HCDR1 comprises the sequence:
SDYAWN (SEQ ID NO:63);
(ii) the HCDR2 comprises the sequence:
YINFDGGTTYNPSLRG (SEQ ID NO:64);
and
(iii) the HCDR3 comprises the sequence:
FYGAKGTLDY (SEQ ID NO:65).

7. The bispecific antibody molecule according to any one of claims 1 to 6, wherein the first VH chain comprises the amino acid sequence or an amino acid sequence at least 90%, at least 95%, or at least 99% identical to the amino acid sequence:
QVQLQESGPGLVKPSQSLSLTCTVTGFSITSDYAWNWIRQFPGKKLEWMG
YINFDGGTTYNPSLRGRISITRDTSKNQFFLQLRSVTPEDTATYYCATFY
GAKGTLDYWGQGTSVTVSS (SEQ ID NO:5),
EVQLQESGPGLVKPSETLSLTCTVSGFSITSDYAWNWIRQPPGKGLEWMG
YINFDGGTTYNPSLRGRITISRDTSKNQFSLKLSSVTAADTAVYYCATFY
GAKGTLDYWGQGTLVTVSS (SEQ ID NO:6),
or
EVQLQESGPGLVKPSETLSLTCTVSGFSITSDYAWNWIRQPPGKGLEWIG
YINFDGGTTYNPSLRGRVTISRDTSKNQFSLKLSSVTAADTAVYYCATFY
GAKGTLDYWGQGTLVTVSS (SEQ ID NO:7).

8. The bispecific antibody molecule according to any one of the preceding claims, wherein the first and/or second VH chain is humanized and/or the VL chain is humanized.

9. The bispecific antibody molecule according to any one of the preceding claims, wherein the second VH chain is derived from a monospecific antibody molecule which binds the TAA, and wherein the affinity of the bispecific antibody molecule for the TAA when paired with one of the light chains is at least 2-fold lower than the affinity of the monospecific antibody molecule for the TAA from which the VH chain is derived.

10. The bispecific antibody molecule according to any one of the preceding claims, wherein the TAA is CD47.

11. The bispecific antibody molecule according to claim 10, wherein the antigen-binding site of the second VH chain comprises the HCDRs 1-3 of a VH chain comprising the amino acid sequence:
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSS (SEQ ID NO:8).

12. The bispecific antibody molecule according to any one of claims 1 to 11, wherein the second VH chain comprises the HCDR1 comprising the sequence: NYNMH (SEQ ID NO:9), the HCDR2 comprising the sequence: TIYPGNDDTSYNQKFKD (SEQ ID NO:10), and the HCDR3 comprising the sequence: GGYRAMDY (SEQ ID NO:11).

13. The bispecific antibody molecule according to any one of claims 1 to 12, wherein the second VH chain comprises the amino acid sequence or an amino acid sequence at least 90%, at least 95%, or at least 99% identical to the amino acid sequence:
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSS (SEQ ID NO:8),
EVQLVESGGGLVQPGGSLRLSCAASGYTFTNYNMHWVRQAPGKGLEWMGT
IYPGNDDTSYNQKFKDRVTISRDNSKNTLYLQMNSLRAEDTAVYYCARGG
YRAMDYWGQGTLVTVSS (SEQ ID NO:66),
EVQLVQSGAEVKKPGESLKISCKGSGYTFTNYNMHWVRQMPGKGLEWMGT
IYPGNDDTSYNQKFKDQVTISADKSISTAYLQWSSLKASDTAMYYCARGG
YRAMDYWGQGTTVTVSS (SEQ ID NO:67),
or
QVQLVQSGSELKKPGASVKVSCKASGYTFTNYNMHWVRQAPGQGLEWMGT
IYPGNDDTSYNQKFKDRFVFSLDTSVSTAYLQISSLKAEDTAVYYCARGG
YRAMDYWGQGTTVTVSS (SEQ ID NO:68).

14. The bispecific antibody molecule according to any one of claims 1-9, wherein the TAA is receptor tyrosine-protein kinase erbB-1.

15. The bispecific antibody molecule according to claim 14, wherein the antigen-binding site of the second VH chain comprises the HCDRs 1-3 of a VH chain comprising the amino acid sequence:
QVQLQESGPGLVKPSQTLSLTCTVSGGSISSGDYYWSWIRQPPGKGLEWI
GYIYYSGSTDYNPSLKSRVTMSVDTSKNQFSLKVNSVTAADTAVYYCARV
SIFGVGTFDYWGQGTLVTVSS (SEQ ID NO:22).

16. The bispecific antibody molecule according to claim 14, wherein the antigen-binding site of the second VH chain comprises the HCDR1 comprising the sequence: SGDYYWS (SEQ ID NO:19), the HCDR2 comprising the sequence: YIYYSGSTDYNPSLKS (SEQ ID NO:20), and the HCDR3 comprising the sequence: VSIFGVGTFDY (SEQ ID NO:21).

17. The bispecific antibody molecule according to any one of claims 14 to 16, wherein the second VH chain comprises the amino acid sequence or an amino acid sequence at least 90%, at least 95%, or at least 99% identical to the amino acid sequence:
QVQLQESGPGLVKPSQTLSLTCTVSGGSISSGDYYWSWIRQPPGKGLEWI
GYIYYSGSTDYNPSLKSRVTMSVDTSKNQFSLKVNSVTAADTAVYYCARV
SIFGVGTFDYWGQGTLVTVSS (SEQ ID NO:22).

18. The bispecific antibody molecule according to any one of claims 1-9, wherein the TAA is receptor tyrosine-protein kinase erbB-2.

19. The bispecific antibody molecule according to claim 18, wherein the antigen-binding site of the second VH chain comprises the HCDRs 1-3 of a VH chain comprising the amino acid sequence:
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSS (SEQ ID NO:12).

20. The bispecific antibody molecule according to claim 18, wherein the antigen-binding site of the second VH chain comprises the HCDR1 comprising the sequence: DYTMD (SEQ ID NO:13), the HCDR2 comprising the sequence: DVNPNSGGSIYNQRFKG (SEQ ID NO:14), and the HCDR3 comprising the sequence: NLGPSFYFDY (SEQ ID NO:15).

21. The bispecific antibody molecule according to any one of claims 18 to 20, wherein the second VH chain comprises the amino acid sequence or an amino acid sequence at least 90%, at least 95%, or at least 99% identical to the amino acid sequence:
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSS (SEQ ID NO:12).

22. The bispecific antibody molecule according to any one of claims 1-9, wherein the TAA is programmed cell death ligand 1 (PD-L1).

23. The bispecific antibody molecule according to claim 22, wherein the antigen-binding site of the second VH chain comprises the HCDRs 1-3 of a VH chain comprising the amino acid sequence:
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS (SEQ ID NO:27).

24. The bispecific antibody molecule according to claim 22, wherein the antigen-binding site of the second VH chain comprises the HCDR1 comprising the sequence: DSWIH (SEQ ID NO:28), the HCDR2 comprising the sequence: WISPYGGSTYYADSVKG (SEQ ID NO:29), and the HCDR3 comprising the sequence: RHWPGGFDY (SEQ ID NO:30).

25. The bispecific antibody molecule according to any one of claims 22-24, wherein the second VH chain comprises the amino acid sequence or an amino acid sequence at least 90%, at least 95%, or at least 99% identical to the amino acid sequence:
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS (SEQ ID NO:27).

26. The bispecific antibody molecule according to any one of the preceding claims , wherein the antibody comprises a Fc domain that has been modified to reduce or abrogate binding of the antibody to one or more Fcy receptors.

27. The bispecific antibody molecule according to any one of the preceding claims for use in a method of treating cancer in a subject, the method comprising administering the antibody to the subject.

28. The bispecific antibody molecule for use according to claim 26, wherein the method comprises administering the antibody in combination with at least one additional active agent, wherein the at least one additional active agent comprises a chemotherapeutic agent, an inhibitor of a multidrug resistance transporter, an immunotherapy agent, or a combination thereof.

29. The bispecific antibody molecule for use according to claim 27, wherein the at least one additional active agent is a chemotherapeutic agent, optionally wherein the chemotherapeutic agent is a taxol, a vinca alkaloid, an anthracycline, Etoposide, Mitoxantrone, or Methotrexate.

30. A chemotherapy agent for use in a method of treating cancer in a subject, the method comprising administering the chemotherapy agent in combination with the antibody according to any one of claims 1 to 26 to the subject, optionally wherein the chemotherapy agent is a taxol, a vinca alkaloid, or an anthracycline.

31. The bispecific antibody molecule for use according to claims 27-29, wherein the subject being treated has a cancer that has been determined to be resistant to the chemotherapeutic agent.

32. A method of treating a subject for a cancer, the method comprising administering to the subject a therapeutically effective amount of the bispecific antibody molecule according to any of claims 1 to 26.

33. The method according to claim 32, wherein the method comprises administering the bispecific antibody molecule in combination with at least one additional active agent, wherein the at least one additional active agent comprises a chemotherapeutic agent, an inhibitor of a multidrug resistance transporter, an immunotherapy agent, or a combination thereof.

34. The method according to claim 33, wherein the at least one additional active agent is a chemotherapeutic agent, optionally wherein the chemotherapeutic agent is a taxol, a vinca alkaloid, an anthracycline, Etoposide, Mitoxantrone, or Methotrexate.

35. The method according to claim 33, wherein the subject being treated has a cancer that has been determined to be resistant to treatment with the chemotherapeutic agent.

36. A pharmaceutical composition comprising:
the antibody of any one of claims 1-26; and
a pharmaceutically acceptable excipient.

37. The pharmaceutical composition according to claim 36, further comprising at least one additional active agent.

38. The pharmaceutical composition according to claim 37, wherein the at least one additional active agent comprises a chemotherapy agent.

39. The pharmaceutical composition according to claim 38, wherein the chemotherapy agent is a taxol, a vinca alkaloid, an anthracycline, Etoposide, Mitoxantrone, or Methotrexate.

40. The pharmaceutical composition according to any of claims 37 to 39, wherein the at least one additional active agent comprises an inhibitor of a multidrug resistance transporter.

41. The pharmaceutical composition according to claim 40, wherein the at least one additional active agent comprises an immunotherapy agent.

42. One or more nucleic acids comprising one or more sequences encoding the antibody according to any of the preceding claims.

43. The one or more nucleic acid according to claim 42, wherein the one or more sequences are operably linked to a promoter.

44. One or more recombinant expression vectors comprising the one or more nucleic acids according to claim 42 or 43.

45. A mammalian cell genetically modified with the one or more recombinant expression vectors according to claim 44.

46. The cell according to claim 45, wherein the cell is an immune cell.

47. A kit comprising:
the antibody or a nucleic acid encoding the antibody, according to any of claims 1 to 26; and
at least one additional active agent.

48. The kit according to claim 47, wherein the at least one additional active agent comprises a chemotherapy agent, an inhibitor of a multidrug resistance transporter, an immunotherapy agent, or a combination thereof.

49. A method of killing a cancer cell, the method comprising contacting the cancer cell with the antibody according to any of claims 1 to 26.

50. The method according to claim 49, comprising administering at least one additional active agent.

51. The method according to claim 50, wherein the at least one additional active agent comprises a chemotherapy agent.

52. The method according to claim 50 or 51, wherein the method increases the killing of the cancer cell by at least 5% as compared to contacting with the at least one additional active agent alone.

53. The method according to any of claims 49 to 52, wherein the cancer cell is a drug resistant cancer cell.

54. A method of treating a subject for a cancer, the method comprising administering to the subject the antibody according to any of claims 1 to 26 or the pharmaceutical composition according to any of claims 35-41.

55. The method according to claim 54, wherein the subject has been treated previously for the cancer.

56. The method according to claim 54 or 55, wherein the cancer is drug resistant or multidrug resistant.

57. The method according to claim 56, wherein the cancer is resistant to a chemotherapeutic agent.

58. The method according to claim 56, wherein the cancer is resistant to an immunotherapy agent.

59. The method according to any of claims 54 to 56, wherein the cancer is resistant to an inhibitor of a multidrug resistance transporter.

60. The method according to any of claims 54 to 59, further comprising administering at least one additional active agent to the subject.

61. The method according to claim 60, wherein the at least one additional active agent comprises a chemotherapy agent.

62. The method according to claim 61, wherein the chemotherapy agent is a taxol, a vinca alkaloid, an anthracycline, Etoposide, Mitoxantrone, or Methotrexate.

63. The method according to any of claims 60 to 62, wherein the at least one additional active agent comprises an inhibitor of a multidrug resistance transporter.

64. The method according to any of claims 60 to 62, wherein the at least one additional active agent comprises an immunotherapy agent.

65. The method according to any of claims 60 to 62, wherein the method increases the effectiveness of the at least one additional active agent as compared to treatment with the at least one additional active agent alone.

66. The method according to claim 65, wherein the increased effectiveness comprises an at least 5% increase in cancer cell killing.

67. The method according to any of claims 54 to 66, further comprising analyzing a sample of the cancer to determine whether the cancer expresses ABCG2 above a predetermined threshold, a tumor associated antigen (TAA) above a predetermined threshold, or both, wherein optionally the TAA comprises CD47, erbB1, erbB2, or PD-L1.

68. The method according to claim 67, wherein the predetermined threshold corresponds to a level of ABCG2 and/or TAA expressed by a reference cell.

69. The method according to claim 68, wherein ABCG2 and/or TAA has been knocked-out of or knocked-down in the reference cell.

70. The method according to claim 68 or 69, wherein the reference cell is a non-cancerous cell.

71. The method according to claim 68, wherein the non-cancerous cell expresses a normal level of ABCG2 and/or TAA.

72. The method according to any of claims 54 to 71, wherein if the cancer expresses ABCG2 and TAA at or above the predetermined thresholds then the subject is administered the multi-specific antibody, and if the cancer expresses ABCG2 or TAA below the predetermined thresholds then the subject is treated with a conventional therapy without administering the multi-specific antibody.

73. A bispecific antibody molecule that binds ATP Binding Cassette Subfamily G Member 2 (ABCG2) and a tumor associated antigen (TAA), wherein the antibody molecule comprises:
a first VH chain and a first VL chain each comprising an antigen-binding site for ABCG2, wherein the first VH chain comprises HCDRs1-3 of the amino acid sequence set forth in SEQ ID NO:5 and the first VL chain comprises LCDRs1-3 of the amino acid sequence set forth in SEQ ID NO:1; and
wherein the TAA is PD-L1 and the antibody molecule further comprises a second VH chain and a second VL chain, wherein the second VH chain comprises HCDRs1-3 of the amino acid sequence set forth in SEQ ID NO:27 and the second VL chain comprises LCDRs1-3 of the VL chain of an anti-PD-L1 antibody such as Atezolizmumab, or
wherein the TAA is CD47 and the antibody molecule further comprises a second VH chain and a second VL chain, wherein the second VH chain comprises HCDRs1-3 of the amino acid sequence set forth in SEQ ID NO:8 and the second VL chain comprises LCDRs1-3 of the VL chain of an anti-CD47 antibody such as 5F9, or
wherein the TAA is HER2 (ErbB2) and the antibody molecule further comprises a second VH chain and a second VL chain, wherein the second VH chain comprises HCDRs1-3 of the amino acid sequence set forth in SEQ ID NO: 16 and the second VL chain comprises LCDRs1-3 of the VL chain of an anti-HER2 antibody such as pertuzumab or trastuzumab, or
wherein the TAA is HER1 (ErbB1) and the antibody molecule further comprises a second VH chain and a second VL chain, wherein the second VH chain comprises HCDRs1-3 of the amino acid sequence set forth in SEQ ID NO: 17 and a second VL chain comprises LCDRs1-3 of the VL chain of an anti-HER1 antibody such as necitumab, and
wherein the bispecific antibody binds to cancer cells expressing both ABCG2 and the TAA while showing reduced binding to non-cancer cells expressing ABCG2 and/or the TAA.
